(19) United States
(12) Patent Application Publication
(10) Pub. No.: US 2025/0017942 A1
(43) Pub. Date: Jan. 16, 2025

(54) Prevention and treatment of osteoarticular diseases by inhibiting acetylcholinesterase

(71) Applicant: THE HONG KONG POLYTECHNIC UNIVERSITY, Hong Kong (CN)

(72) Inventor: Chunyi WEN, Hong Kong (CN)

(21) Appl. No.: 18/705,620

(22) PCT Filed: Oct. 27, 2021
(86) PCT No.: PCT/CN2021/126593

§ 371 Date: Apr. 29, 2024

(51) Int. Cl. A61K 31/55; A61K 31/165; A61K 31/439; A61K 31/445; A61K 31/473; A61P 19/02

(52) U.S. Cl. CPC A61K 31/55; A61K 31/165; A61K 31/439; A61K 31/445; A61K 31/473; A61P 19/02

(57) ABSTRACT

The present application relates to the prevention and treatment of osteoarticular diseases, in particular osteoarthritis (OA), by inhibition of acetylcholinesterase (AChE). Specifically, this application provides a disease progression-modifying drug for the treatment of osteoarthritis, i.e., an acetylcholinesterase inhibitor, in particular a dimeric acetylcholinesterase inhibitor. The dimer targets both the enzymatic triad and non-enzymatic peripheral sites of acetylcholinesterase, thereby enhancing protection of articular cartilage and subchondral bone.

### TECHNICAL FIELD

The present application relates to the field of prevention and treatment of osteoarticular diseases, in particular osteoarthritis (OA). Specifically, the present application relates to the prevention and treatment of osteoarticular diseases, especially osteoarthritis, by inhibition of acetylcholinesterase.

### BACKGROUND

Osteoarticular diseases may include a variety of types, such as degenerative arthritis, bursitis, synovitis, cervical spondylosis, lumbar spondylosis, scapulohumeral periarthritis, hyperosteogeny, rheumatic arthritis, rheumatoid arthritis, and the like.

Osteoarthritis (OA), also known as degenerative arthritis, is a degenerative disease that is degradation of articular cartilage, reactive hyperplasia of articular margins and subchondral bone due to factors such as aging, obesity, strain, trauma, congenital anomalies of joints, joint deformities, and the like. Osteoarthritis is the most common degenerative joint disorder, mainly affecting weight-bearing joints such as hips and knees, and is a major cause of physical disability, clinically manifested by slow-developing joint pain, tenderness, stiffness, joint swelling, limited mobility, and joint deformity, among others. Despite the identification of risk factors such as mechanistic, metabolic or genetic, the exact pathogenesis for osteoarthritis remains unclear.

The current treatment and prevention for OAis non-specific, including a non-pharmaceutical approach, such as through body weight control, increased exercise and injury prevention, and a pharmacological approach using different forms of pain medications. Most of the recommended drugs are topical and oral non-steroidal anti-inflammatory drugs (NSAIDs), but their analgesic effects last only for a few days to months. Other options are intra-articular injection of hyaluronic acid or glucocorticoids. The role of chondroitin and glucosamine in treating OA is still being debated. A meta-analysis of controlled randomized trials detected that oral chondroitin was more effective than placebo in relieving pain, while glucosamine was more effective compared to placebo in reducing stiffness. However, all these treatment options are only able to temporarily relieve joint pain and cannot alter the course of disease progression. In the advanced stage of disease, patients have to receive surgical treatment, either fixing cartilage lesion under arthroscopy or replacing the damaged joint with an artificial metal with significant co-morbidities and complications.

Thus, there is currently no effective disease modifying treatment for osteoarthritis until the end stage of disease necessitating joint replacement. There is an urgent need in the art for a novel drug that alters the disease progression of osteoarthritis to prevent or treat osteoarthritis.

### SUMMARY

The technical solution of the present application is based, at least in part, on the inventors' first discovery that the expression of acetylcholinesterase (AChE) is increased in chondrocytes of osteoarthritis patients compared to normal healthy populations. This discovery indicates a role of acetylcholinesterase in the development of osteoarthritis (i.e., the involvement of acetylcholinesterase in the disease progression of osteoarthritis) and the feasibility of acetylcholinesterase inhibitors as a treatment option for osteoarthritis (OA).

The novel osteoarthritis disease-modifying drugs, dimeric acetylcholinesterase inhibitors used in present application target both the enzymatic triad and non-enzymatic peripheral sites of acetylcholinesterase. This dual blockade of acetylcholinesterase is established by dimerization of available drugs.

The present application envisages and validates the use of acetylcholinesterase inhibitors, in particular acetylcholinesterase inhibitors based on the dimer of tacrine and huperzine A, for the prevention or treatment of osteoarthritis. Specifically, the inventors of the present application have also demonstrated the effects of acetylcholinesterase inhibitors on osteoblasts and osteoclasts, on the inflammatory activation of chondrocyte and on oxidative stress-induced activation of chondrocytes.

In a first aspect, the present application provides a method of treating an osteoarticular disease in a subject in need thereof, comprising administering to the subject a therapeutically effective amount of an acetylcholinesterase inhibitor. In some embodiments, the osteoarticular disease is an osteoarticular disease associated with increased expression of acetylcholinesterase. In some embodiments, the osteoarticular disease includes osteoarthritis, rheumatic arthritis, rheumatoid arthritis, bursitis, synovitis, cervical spondylosis, lumbar spondylosis, scapulohumeral periarthritis, hyperosteogeny, ligament injury and local joint inflammation. In some embodiments, the osteoarticular disease is osteoarthritis. In some embodiments, the osteoarticular disease is synovitis. In some embodiments, the osteoarticular disease is ligament injury.

In a second aspect, the present application relates to the use of an acetylcholinesterase inhibitor in the prevention or treatment of an osteoarticular disease. In some embodiments, the osteoarticular disease is an osteoarticular disease associated with increased expression of acetylcholinesterase. In some embodiments, the osteoarticular disease includes osteoarthritis, rheumatic arthritis, rheumatoid arthritis, bursitis, synovitis, cervical spondylosis, lumbar spondylosis, scapulohumeral periarthritis, hyperosteogeny, ligament injury and local joint inflammation. In some embodiments, the osteoarticular disease is osteoarthritis. In some embodiments, the osteoarticular disease is synovitis. In some embodiments, the osteoarticular disease is ligament injury.

In a third aspect, the present application relates to the use of an acetylcholinesterase inhibitor for one or more of:
providing osteoprotection;
improving bone structure;
maintaining bone homeostasis;
reducing inflammatory activation of chondrocytes;
providing an anti-catabolic and/or pro-anabolic effect on chondrocytes;
reducing telomere length; and
reducing oxidative stress-induced senescence.

In a fourth aspect, the present application relates to the use of an acetylcholinesterase inhibitor in preparation of a drug for the prevention or treatment of an osteoarticular disease. In some embodiments, the osteoarticular disease is an osteoarticular disease associated with increased expression of acetylcholinesterase. In some embodiments, the osteoarticular disease includes osteoarthritis, rheumatic arthritis, rheumatoid arthritis, bursitis, synovitis, cervical spondylosis, lumbar spondylosis, scapulohumeral periarthritis, hyperosteogeny, ligament injury and local joint inflammation. In some embodiments, the osteoarticular disease is osteoarthritis. In some embodiments, the osteoarticular disease is synovitis. In some embodiments, the osteoarticular disease is ligament injury.

In a fifth aspect, the present application relates to the use of an acetylcholinesterase inhibitor in preparation of a drug for one or more of:
providing osteoprotection;
improving bone structure;
maintaining bone homeostasis;
reducing inflammatory activation of chondrocytes;
providing an anti-catabolic and/or pro-anabolic effect on chondrocytes;
reducing telomere length; and
reducing oxidative stress-induced senescence.

In some embodiments of each of the above aspects, the acetylcholinesterase inhibitor is selected from 7-methoxytacrine, huperzine A, donepezil, galartamine, ambenonium chloride, and the like. Preferably, the acetylcholinesterase inhibitor is donepezil.

In some embodiments of each of the above aspects, the acetylcholinesterase inhibitor is a dimeric acetylcholinesterase inhibitor. In some embodiments, the dimeric acetylcholinesterase inhibitor may have the form of formula A-L-B, wherein A and B are acetylcholinesterase inhibitor monomers and may be the same or different, and L is an optional linker. In some embodiments, L, when present, is selected from alkylene and —(CH₂—CH₂—NH—)n—CH₂—CH₂—, wherein n=1-5, e.g., 1, 2, 3, 4, 5. And preferably, L is an alkylene group-(CH₂)n—, wherein n is an integer from 1 to 20. In some embodiments, A and B are independently selected from the group consisting of tacrine and huperzine A. Preferably, the linker L connects A and B through amino groups on A and B.

In some further embodiments, the dimeric acetylcholinesterase inhibitor may be a homodimer or heterodimer. Preferably, the dimeric acetylcholinesterase inhibitor is selected from a homodimer or heterodimer of tacrine and huperzine A.

In some embodiments, the dimeric acetylcholinesterase inhibitor is a tacrine homodimer, i.e., bis(n)-tacrine (also known as bis(n)-Cognex, abbreviated as B(n)C), as shown below:

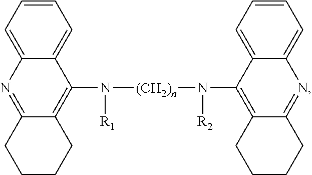

wherein R₁ and R₂ are each independently H or C₁₋₄alkyl, and n=2-10.

Preferably, R₁ and R₂ are the same, and more preferably, both are H.

More preferably, the tacrine homodimer is B(3)C or B(7)C, i.e., N=3 or 7: B(3)-C B(7)-C

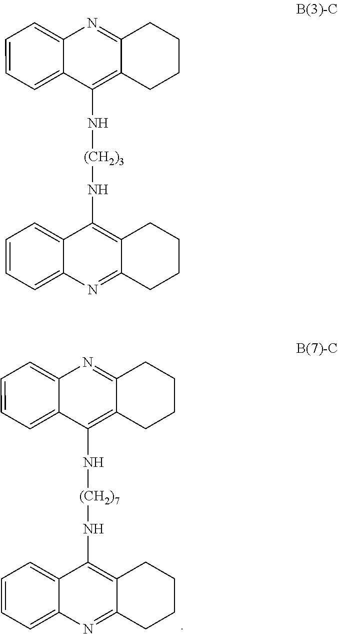

In some embodiments, the dimeric acetylcholinesterase inhibitor is a huperzine A (HA)-tacrine heterodimer as shown below:

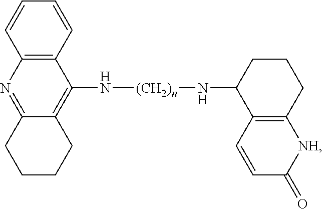

wherein n=4-12.

Preferably, n=10, i.e., the dimer is a huperzine A (HA)-tacrine heterodimer (abbreviated as A10E) as shown below:

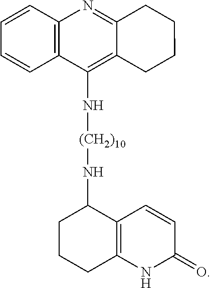

In some embodiments, the dimeric acetylcholinesterase inhibitor is a huperzine A (HA) homodimer having the formula:

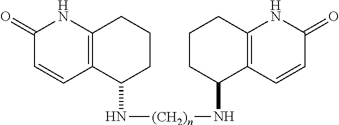

wherein n=10-14.

Preferably, n=12, i.e., the dimer is bis(12)-hupyridone (abbreviated as E12E) as shown below:

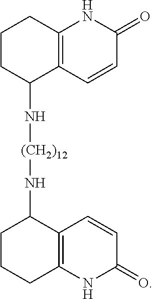

In some embodiments, the acetylcholinesterase inhibitor of the present application is formulated in the form of a pharmaceutical composition comprising a therapeutically effective amount of an acetylcholinesterase inhibitor and, optionally, a pharmaceutically acceptable carrier, such as a diluent, adjuvant, excipient or vehicle. In some embodiments, the pharmaceutical composition is in the form of a solution, suspension, emulsion, tablet, pill, capsule, powder, sustained-release formulation. In some embodiments, the pharmaceutical composition is administered by a route selected from: oral, parenteral, subcutaneous, intramuscular, intravenous, intrarticular, intracapsular, intracartilaginous, intracavitary, intracelial, intraosteal, intrapelvic, intraspinal, intrasynovial, intrathoracic, bolus, buccal, sublingual, intranasal, iontophoretic means, or transdermal means. More preferably, the pharmaceutical composition is administered by intra-articular, oral administration or injection. In some embodiments, the pharmaceutical composition may also comprise other therapeutic agents, e.g., TGF-beta inhibitors, IL-1 inhibitors, corticosteroids, hyaluronic acid, or combinations thereof, and the like.

Specifically, the present application provides the following solutions:

1. A method of preventing or treating an osteoarticular disease in a subject in need thereof, comprising administering to the subject a therapeutically effective amount of an acetylcholinesterase inhibitor.

2. The method according to solution 1, wherein the osteoarticular disease is selected from the group comprising osteoarthritis, rheumatic arthritis, rheumatoid arthritis, bursitis, synovitis, cervical spondylosis, lumbar spondylosis, scapulohumeral periarthritis, hyperosteogeny, ligament injury and local joint inflammation.

3. The method according to solution 2, wherein the osteoarticular disease is selected from osteoarthritis, synovitis, hyperosteogeny, and ligament injury.

4. The method according to solution 3, wherein the osteoarticular disease is osteoarthritis.

5. The method according to solution 1, wherein the acetylcholinesterase inhibitor is selected from the group consisting of: 7-methoxytacrine, huperzine A, donepezil, galantamine and ambenonium chloride.

6. The method according to solution 5, wherein the acetylcholinesterase inhibitor is donepezil.

7. The method according to solution 1, wherein the acetylcholinesterase inhibitor is a dimeric acetylcholinesterase inhibitor.

8. The method according to solution 7, wherein the dimeric acetylcholinesterase inhibitor has the structure of formula A-L-B, wherein A and B are acetylcholinesterase inhibitor monomers and may be the same or different, L is an optional linker and, when present, is preferably an alkylene —(CH₂)n—, wherein n is an integer from 1 to 20, e.g., 2, 3, 4, 5, 6,7, 8, 9, 10, 11, 12, 13, 14, 15, 16, 17, 18, and 19.

9. The method according to solution 8, wherein A and B are independently selected from tacrine and huperzine A.

10. The method according to solution 9, wherein the dimeric acetylcholinesterase inhibitor is a tacrine homodimer, i.e., bis(n)-tacrine, as shown below:

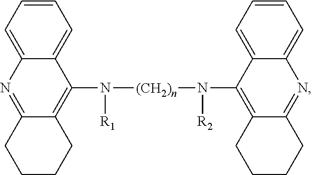

wherein R₁ and R₂ are each independently H or C₁₋₄alkyl, and n=2-10.

11. The method according to solution 10, wherein R₁ and R₂ are both H.

12. The method according to solution 10 or 11, wherein n=3 or 7.

13. The method according to solution 9, wherein the dimeric acetylcholinesterase inhibitor is a huperzine A-tacrine heterodimer, i.e., huperzine A-(n)-tacrine, as shown below:

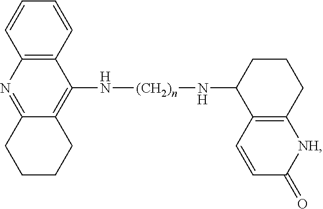

wherein n=4-12.

14. The method according to solution 13, wherein n=10.

15. The method according to solution 9, wherein the dimeric acetylcholine sterase inhibitor is a huperzine A homodimer as shown below:

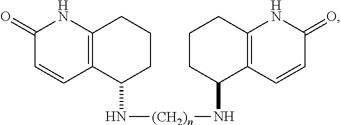

wherein n=10-14.

16. The method according to solution 15, wherein n=12.

17. The method according to solution 9, wherein the dimeric acetylcholinesterase inhibitor is

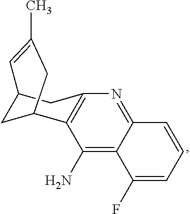 [SMILES: CC1=CC2Cc3nc4cccc(F)c4c(N)c3C(C1)C2]

18. Use of an acetylcholinesterase inhibitor in preparation of a drug for the prevention or treatment of an osteoarticular disease.

19. Use according to solution 18, wherein the osteoarticular disease is selected from the group comprising osteoarthritis, rheumatic arthritis, rheumatoid arthritis, bursitis, synovitis, cervical spondylosis, lumbar spondylosis, scapulohumeral periarthritis, hyperosteogeny, ligament injury and local joint inflammation.

20. Use according to solution 19, wherein the osteoarticular disease is selected from osteoarthritis, synovitis, hyperosteogeny, and ligament injury.

21. Use according to solution 20, wherein the osteoarticular disease is osteoarthritis.

22. Use of an acetylcholinesterase inhibitor in preparation of a drug for one or more of:
providing osteoprotection;
improving bone structure;
maintaining bone homeostasis;
reducing inflammatory activation of chondrocytes;
providing an anti-catabolic and/or pro-anabolic effect on chondrocytes;
reducing telomere length; and
reducing oxidative stress-induced senescence.

23. The use according to any one of solutions 18-22, wherein the acetylcholinesterase inhibitor is selected from the group consisting of: 7-methoxytacrine, huperzine A, donepezil, galantamine and ambenonium chloride.

24. The use according to solution 23, wherein the acetylcholinesterase inhibitor is donepezil.

25. The use according to any one of solutions 18-22, wherein the acetylcholinesterase inhibitor is a dimeric acetylcholinesterase inhibitor.

26. The use according to solution 25, wherein the dimeric acetylcholinesterase inhibitor has the structure of formula A-L-B, wherein A and B are acetylcholinesterase inhibitor monomers and may be the same or different, L is an optional linker and, when present, is preferably an alkylene —(CH₂)n—, wherein n is an integer from 1 to 20, e.g., 2, 3, 4, 5, 6, 7, 8, 9, 10, 11, 12, 13, 14, 15, 16, 17, 18, and 19.

27. The use according to solution 26, wherein A and B are independently selected from tacrine and huperzine A.

28. Use according to solution 27, wherein the dimeric acetylcholinesterase inhibitor is a tacrine homodimer, i.e., bis(n)-tacrine, as shown below:

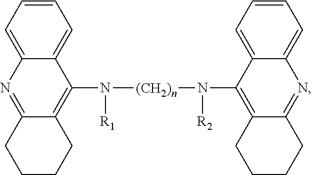

wherein R₁ and R₂ are each independently H or C₁₋₄alkyl, and n=2-10.

29. Use according to solution 28, wherein R₁ and R₂ are both H.

30. The use according to solution 28 or 29, wherein n=3 or 7.

31. The use according to solution 27, wherein the dimeric acetylcholinesterase inhibitor is a huperzine A-tacrine heterodimer, i.e., huperzine A-(n)-tacrine, as shown below:

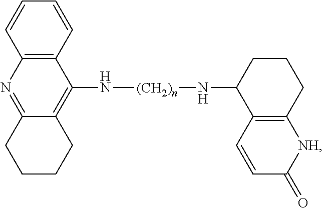

wherein n=4-12.

32. The use according to solution 31, wherein n=10.

33. Use according to solution 27, wherein the dimeric acetylcholinesterase inhibitor is a huperzine A homodimer as shown below:

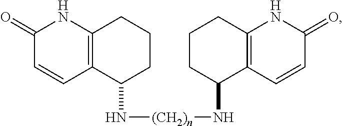

wherein n=10-14.

34. The use according to solution 33, wherein n=12.

35. Use according to solution 27, wherein the dimeric acetylcholinesterase inhibitor is

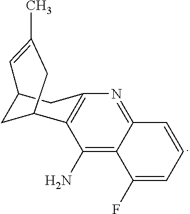 [SMILES: CC1=CC2Cc3nc4cccc(F)c4c(N)c3C(C1)C2]

36. A pharmaceutical composition for preventing or treating an osteoarticular disease comprising a therapeutically effective amount of an acetylcholinesterase inhibitor and a pharmaceutically acceptable carrier.

37. The pharmaceutical composition according to solution 36, wherein the osteoarticular disease is selected from the group comprising osteoarthritis, rheumatic arthritis, rheumatoid arthritis, bursitis, synovitis, cervical spondylosis, lumbar spondylosis, scapulohumeral periarthritis, hyperosteogeny, ligament injury and local joint inflammation.

38. The pharmaceutical composition according to solution 37, wherein the osteoarticular disease is selected from osteoarthritis, synovitis, hyperosteogeny, and ligament injury.

39. The pharmaceutical composition according to solution 38, wherein the osteoarticular disease is osteoarthritis.

40. The pharmaceutical composition according to solution 36, wherein the acetylcholinesterase inhibitor is selected from the group consisting of: tacrine, huperzine A, donepezil, galantamine and ambenonium chloride.

41. The pharmaceutical composition according to solution 40, wherein the acetylcholinesterase inhibitor is donepezil.

42. The pharmaceutical composition according to solution 36, wherein the acetylcholinesterase inhibitor is a dimeric acetylcholinesterase inhibitor.

43. The pharmaceutical composition according to solution 42, wherein the dimeric acetylcholinesterase inhibitor has the structure of formula A-L-B, wherein A and B are acetylcholinesterase inhibitor monomers and may be the same or different, L is an optional linker and, when present, is preferably an alkylene —(CH₂)n—, wherein n is an integer from 1 to 20, e.g., 2, 3, 4, 5, 6, 7, 8, 9, 10, 11, 12, 13, 14, 15, 16, 17, 18, and 19.

44. The pharmaceutical composition according to solution 43, wherein A and B are independently selected from tacrine and huperzine A.

45. The pharmaceutical composition according to solution 44, wherein the dimeric acetylcholinesterase inhibitor is a tacrine homodimer, i.e., bis(n)-tacrine, as shown below:

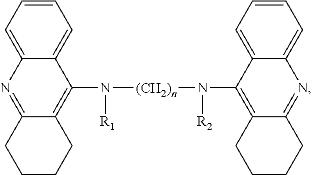

wherein R₁ and R₂ are each independently H or C₁₋₄alkyl, and n=2-10.

46. The pharmaceutical composition according to solution 45, wherein R₁ and R₂ are both H.

47. The pharmaceutical composition according to solution 45 or 46, wherein n=3 or 7.

48. The pharmaceutical composition according to solution 44, wherein the dimeric acetylcholinesterase inhibitor is a huperzine A-tacrine heterodimer, i.e., huperzine A-(n)-tacrine, as shown below:

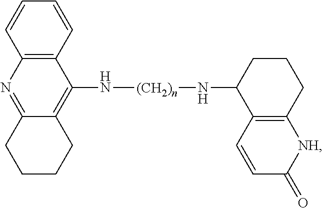

wherein n=4-12.

49. The pharmaceutical composition according to solution 48, wherein n=10.

50. The pharmaceutical composition according to solution 44, wherein the dimeric acetylcholinesterase inhibitor is a huperzine A homodimer as shown below:

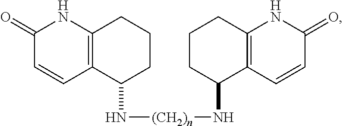

wherein n=10-14.

51. The pharmaceutical composition according to solution 50, wherein n=12.

52. The pharmaceutical composition according to solution 44, wherein the dimeric acetylcholinesterase inhibitor is

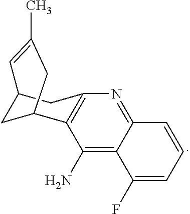 [SMILES: CC1=CC2Cc3nc4cccc(F)c4c(N)c3C(C1)C2]

### BRIEF DESCRIPTION OF THE DRAWINGS

Embodiments of the present application and preferred and advantageous aspects thereof will be better understood when viewed in conjunction with the following drawings, in which:

FIG. 1 compares AChE expression between healthy and OA chondrocytes. This Figure shows a higher expression of AchE in human OA chondrocytes than normal chondrocytes differentiated from human bone marrow mesenchymal stem cells (hMSCs).

FIG. 2 shows DAB staining of acetylcholinesterase in WKY rats, SHR rats and donepezil-treated SHR rats.

FIG. 3 shows the effects of exogenous IL-1β alone or in combination with acetylcholinesterase inhibitors (A10E, E12E) on inflammation (IL-1α, IL-6, TNF-α) and catabolic (MMP13), anabolic (Agg, Sox9, Col2a1) and senescence (p16) markers.

FIG. 4 shows the effects of exogenous hydrogen peroxide (H₂02) alone or in combination with acetylcholinesterase inhibitors (D=donepezil) on AChE mRNA (A) and telomere length (B).

FIG. 5 shows that AChE expression increased with osteoclastogenesis. A) RANKL induced differentiation of murine macrophages (RAW 264.7 cells) towards multinucleated osteoclasts in vitro; B) AChE mRNA expression levels increased along with other osteoclast differentiation mRNA markers (TRAP, Ctsk, MMP9, RNAK); C) Immunofluorescence images showed that AChE protein levels also increased during osteoclastogenesis.

FIG. 6 shows that intact or heat-inactivated recombinant mouse AChE invariably promoted macrophage fusion and osteoclastogenesis. A) Both intact and heat-inactivated recombinant mouse AChE stimulated cell-cell fusion and enlargement after RANKL-induced osteoclastogenesis. B) There was no significant difference between intact and heat-inactivated recombinant mouse AChE in terms of the average nuclear number in single TARP+cell. C) The effects of AChE on cell-cell fusion were associated with an increment of cell fusion proteins, DC-STAMP and OC-STAMP.

FIG. 7 shows that donepezil inhibited cell-cell fusion of osteoclast precursors.

FIG. 8 shows donepezil rescued OVX (ovariectomy)-induced bone loss. Balb/C mice were randomly divided into 4 groups to receive the following treatments: 1) Sham group: Sham surgery+normal saline; 2) OVX group: bilateral OVX+normal saline; 3) Low dose Donepezil group: Bilateral OVX+0.2 mg donepezil/kg body weight, treating starting one month after OVX and continuing for 1 more month; 4) High dose Donepezil group: Bilateral OVX+2 mg donepezil/kg body weight, treating starting one month after OVX and continuing for 1 more month. All animals were sacrificed at 2 months after OVX for mirco-CT and histological examination. The mirco-CT images of primary spongiosa of tibia and vertebral body of lumbar spine showed that donepezil exhibited osteoprotection in a dose-dependent manner.

FIG. 9 shows that donepezil, rather galantamine, suppressed osteoclastogenesis in a dose-dependent manner. Such discrepancy may arise from their different binding patterns in 3D structure of AChE protein. Donepezil occupies the entire binding pocket of AChE from the catalytic triad at the gorge to the peripheral anionic site whereas galantamine selectively binds to the catalytic site of AChE.

FIG. 10 shows the anti-catabolic effects of the FDA approved drugs and the dimers of the present application in vitro. A-H) Both donepezil and the dimers of the present application significantly suppressed osteoclastogenic differentiation of RAW 264.7 cells but galantamine did not. I) The comparisons of anti-catabolic effects on osteoclastogenesis among FDA-approved drugs, alendronate, donepezil, galantamine and dimeric AChE inhibitors of the present application. In terms of the mRNA expression of MMP9, RANK, TRAP, the markers for osteoclasts differentiation and maturation, donepezil and the dimeric AChE inhibitors of the present application are comparable and superior to alendronate.

FIG. 11 The time-course changes of AChE activity and expression during bone development and postnatal growth from embryo (E) to postnatal till 6 months old (A, B). AChE exists in osteoblasts as a proline-rich membrane anchor-linked tetrameric globular form (B). It increases in parallel to osteoblasts differentiation (C).

FIG. 12 shows that the FDA-approved and novel dimeric AChE inhibitors herein provided additional osteoprotection compared to alendronate (with anti-catabolic effect only) although their effect was not so strong as parathyroid hormone (PTH) in vitro. Osteoclast differentiation and mineralization from mouse MSCs was evaluated using Alizarin red staining. All AChE inhibitors demonstrated anti-anabolic effects in a dose-dependent manner. The promotive effects of donepezil and bis(3)-tacrine (B3C) were observed at the concentration of 1 μM although it was not so strong as PTH.

### DETAILED DESCRIPTION OF THE INVENTION

It is understood that this application is not limited to the specific methods and components, etc., described herein, as these may vary. It is also to be understood that the terminology used herein is used for the purpose of describing particular embodiments only, and is not intended to limit the scope of the invention.

As used in this specification and the claims, the singular forms “a,” “an,” and “the” include plural reference unless the context clearly dictates otherwise.

Unless defined otherwise, all technical and scientific terms used herein have the same meaning as commonly understood by one of ordinary skill in the art to which this invention belongs. Specific methods, compositions, and materials are described, although it is understood that any materials and methods similar or equivalent to those described herein can also be used in the practice or testing of the regimes of the present application.

### Acetylcholinesterases

Acetylcholinesterase, AChE for short, is a key enzyme in biological nerve conduction that, between cholinergic synapses, can degrade acetylcholine, terminate the excitatory effect of neurotransmitters on the postsynaptic membrane, and ensure the normal transmission of nerve signals within the organism. With carboxypeptidase and aminopeptidase activity, acetylcholinesterase is involved in cell development and maturation, and promotes neuronal development and nerve regeneration.

AChE is a serine hydrolase mainly found at neuromuscular junctions and cholinergic brain synapses. Its principal biological role is termination of impulse transmission at cholinergic synapses by rapid hydrolysis of the neurotransmitter acetylcholine (Ach) to acetate and choline. AChE has a remarkably high specific catalytic activity—each molecule of AChE degrades about 25000 molecules of acetylcholine (ACh) per second.

AChE (see, e.g., Li, Wenming, et a1. “Novel anti-Alzheimer's dimer bis(7)-cognitin: cellular and molecular mechanisms of neuroprotection through multiple targets.” Neurotherapeutics 6.1 (2009): 187-201, and Sussman, Joel L., et a1. “Atomic structure of acetylcholinesterase from Torpedo californica: a prototypic acetylcholine-binding protein.” Science 253.5022 (1991): 872-879) has an ellipsoidal shape with dimensions ˜45 Å×60 Å×65 Å consisting of 12-stranded central mixed β-sheet surrounded by 14 α helices. The first and last pair of strands each form β-hairpin loops that are only loosely hydrogen-bonded to the other eight central, super-helically twisted strands. The active site of AChE is formed by two subsites: the “esteratic” site (ES) and the “anionic” site (AS). These two sites are believed to be responsible for the catalytic machinery and the choline-binding pocket of the enzyme, respectively. By sequence alignment and site-directed mutagenesis, Ser²⁰⁰, His⁴⁴⁰ and Glu³²⁷ are found to form a planar array in the active site that resembles the catalytic triad of chymotrypsin and other serine proteases.

In addition to the catalytic site, AChE possesses another subsite that is able to bind acetylcholine (Ach) or quaternary ligands. Using photo-label and affinity label, the sequences of peptides of residues 251-264 and peptides of residues 270-278 have been identified as peripheral binding sequences. These two peptides lie on the surface of the protein near the rim of the enzyme gorge and bind the bis-quaternary ligands, which usually serve as inhibitors, at one end to the peripheral binding site and the other end to the aromatic lining of the enzyme pocket. Because this subsite is distinct from the other two catalytic subsites, it is named the “peripheral” site and is a non-competitive binding site. The two catalytic sites, together with the third peripheral site, make up the active site gorge of the enzyme (see following figure).

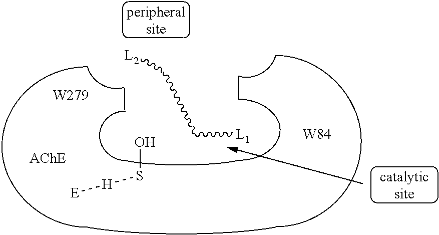

The peripheral site provides aromatic guidance, which involves three conserved aromatic residues: Tyr⁷⁰, Trp²⁷⁹ and Tyr¹²¹, which increase the concentration of Ach at the opening of the active site gorge, facilitating passage through the narrower portion of the gorge toward the catalytic site. Once the ligands are trapped at the top of the enzyme gorge, they can easily diffuse into the active site deep in the gorge. Structurally, this enzyme gorge is a deep and narrow channel extending from the surface to halfway inside of the enzyme (˜20 Å long). The gorge is so deep that it can bind many different substrates, agonists, and inhibitors.

### Acetylcholinesterase Inhibitors

AChE inhibitors inhibit the cholinesterase enzyme from breaking down ACh, increasing both the level and duration of neurotransmitter action. According to the mode of action, AChE inhibitors can be divided into two groups: irreversible inhibitors and reversible inhibitors. Reversible inhibitors, competitive or noncompetitive, may have therapeutic applications, while toxic effects are associated with irreversible AChE activity modulators.

### i) Reversible Acetylcholinesterase Inhibitors

Reversible acetylcholinesterase inhibitors play an important role in pharmacological manipulation of the enzyme activity. These inhibitors include compounds with different functional groups (carbamate, quaternary or tertiary ammonium group), and have been applied in the diagnostic and/or treatment of various diseases such as: myasthenia gravis, Alzheimer's disease (AD), postoperative ileus, bladder distention, glaucoma, as well as antidote to anticholinergic overdose.

### 1) Reversible Acetylcholinesterase Inhibitors Used in Treatment of Alzheimer's Disease

### Donepezil

Donepezil is a selective, reversible AChE inhibitor that binds to the peripheral anionic sites. This medicine, a reversible cholinesterase inhibitor approved by the US FDA for marketing in 1996 for the treatment of mild-to-moderate AD, is a piperidine derivative, which is highly selective for acetylcholinesterase of the central nervous system, with a selective affinity for acetylcholinesterase that is 1250 times more potent than for butyrylcholinesterase, and therefore has no significant peripheral cholinergic effects and less side effects.

### Rivastigmine:

Rivastigmine, sold under the trade name Exelon, is a powerful, slowly reversible carbamate inhibitor that blocks cholinesterase activity through binding at the esteratic moiety of the active site. Unlike donepezil that selectively inhibits AChE, rivastigmine inhibits both BuChE (butyrylcholinesterase) and AChE.

### Galantamine

Galantamine, sold under the trade name Razadyne, Nivalin, is an alkaloid isolated from the plant Galanthus woronowii being applied for mild-to-moderate AD. It is a selective, competitive, rapidly-reversible AChE inhibitor that interacts with the anionic subsites, as well as with the aromatic gorge. It interacts with the nicotinic receptor at binding sites separate from those for ACh and nicotinic agonists, and acts specifically to enhance the activity (sensitize) of nicotinic receptors in the presence of ACh.

### 7-Methoxytacrine

7-Methoxytacrine was widely studied as a suitable substitute to tacrine. In vitro and in vivo tests indicated both its less toxic effects and stronger inactivating power against AChE related to tacrine.

### Huperzine A

Huperzine A is an alkaloid extracted from Huperzia serrata, which was approved for marketing in 1994. It is a highly selective, competitive and non-competitive, mixed inhibitor of cholinesterase, and its target is the peripheral anion site.

### Ambenonium Chloride

Amberium chloride, as an anticholinesterase drug, possesses anticholinesterase effect and skeletal muscle excitation, mainly used for intestinal flatulence and myasthenia gravis etc.

The structures of donepezil, rivastigmine, galantamine, tacrine, 7-methoxytacrine, and huperzine A are shown below:

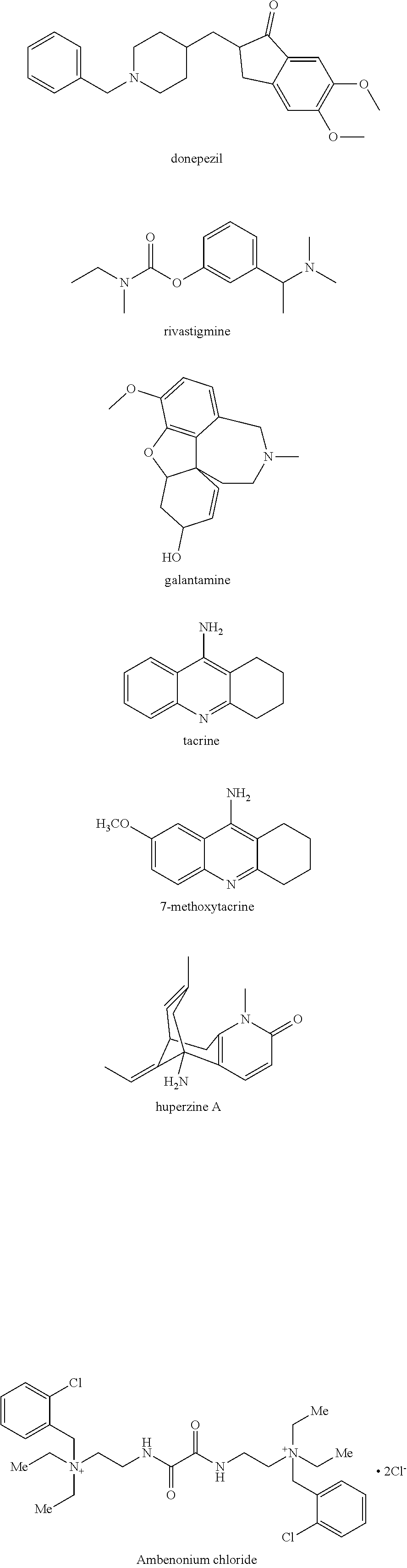

### 2) Carbamates

Carbamates are organic compounds derived from carbamic acid (NH₂COOH), and the structure of biologically active carbamates is displayed below:

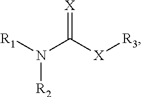

wherein X can be oxygen or sulfur (thiocarbamate), R₁ and R₂ are usually organic or alkyl substituents, but R₁ or R₂ may also be hydrogen, and R₃ is mostly an organic substituent or sometimes a metal.

Carbamates, due to their reversible AChE inhibitory action, found an important application in human medicine as pharmacologically active compounds. Natural carbamate derivate physostigmine, the secondary metabolite in the plant Physostigma venenosum, is widely used in the treatment of myasthenia gravis. As a potent AChE inhibitor, this therapeutic agent reduces ACh hydrolysis rate, and thereby increases its level in damaged neurosynaptic clefts improving nerve impulse transmission. In addition, physostigmine is capable to prevent irreversible binding of OP to AChE. Consequently, it is applied as a prophylactic against nerve agent intoxication. Furthermore, rivastigmine is a carbamate with probably the most meaningful pharmacological application, validated in the symptomatic treatment of AD, as described above.

### ii) Irreversible Acetylcholinesterase Inhibitors-Organophosphorus Compounds (OP)

OP are esters or thiols derived from phosphoric, phosphonic, phosphinic or phosphoramidic acid, having the structure shown below:

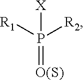

R₁ and R₂ are aryl or alkyl groups that are bonded to the phosphorus atom either directly (forming phosphinates), or through an oxygen or sulphur atom (forming phosphates or phosphothioates). In some cases, R₁ is directly bonded to the phosphorus atom, and R₂ is bonded to an oxygen or sulphur atom (forming phosphonates or thiophosphonates). In phosphoramidates, at least one of these groups is —NH₂ (un-, mono- or bi-substituted), and the atom double-bonded with phosphorus is either oxygen or sulphur. The X group, also binding to the phosphorus atom through oxygen or sulphur atom, may belong to a wide range of halogen, aliphatic, aromatic or heterocyclic groups. This “leaving group” is released from the phosphorus atom when OP is hydrolyzed by phosphotriesterases or upon interaction with protein targets.

OP exert their main toxicological effects through non-reversible phosphorylation of esterases in the central nervous system. The acute toxic effects are related to irreversible inactivation of AChE. Actually, OPs are substrate analogues to ACh, and like natural substrate enter the active site covalently binding to serine —OH group. As in acetylation, OP is split and the enzyme is phosphorylated. While the acyl enzyme is quickly hydrolyzed to regenerate the free enzyme, dephosphorylation is very slow (several days), and phosphorylated enzyme cannot hydrolyze the neurotransmitter.

### Dimeric Acetylcholinesterase Inhibitors

In one aspect, the inhibitor useful in the present disclosure is in the form of a dimer. In some embodiments, the dimeric acetylcholinesterase inhibitor may have the form of formula A-L-B, wherein A and B are acetylcholinesterase inhibitor monomers and may be the same or different, and L is an optional linker. In some embodiments, L, when present, is selected from alkylene and —(CH₂—CH₂—NH—)n—CH₂—CH₂—, wherein n=1-5, e.g., 1, 2, 3, 4, 5. And preferably, L is an alkylene group-(CH₂)n—, wherein n is an integer from 1 to 20. In one embodiment, A and B are independently selected from tacrine, a previously FDA approved AChE inhibitor, and huperzine A (HA), a potent AChE inhibitor originally isolated from the Chinese herbal medicine Huperzia serrata (commonly known as Qian Ceng Ta). Preferably, the linker L connects A and B through amino groups on A and B.

Tacrine, a weakly basic compound, with the chemical name tetrahydroaminoacridine and trade name Cognex®, is the first acetylcholinesterase inhibitor used to treat Alzheimer's disease. Tacrine can effectively suppress acetylcholine breakdown within the brain, increase the choline level in the cerebral cortex, improve the metabolic function of the brain, and moderately relieve Alzheimer's disease symptoms. Tacrine was approved for marketing by the U.S. FDA in 1993, mainly for clinical application in the treatment of patients with mild-to-moderate Alzheimer's disease.

Huperzine A is a natural plant alkaloid and is a potent, reversible, highly selective second-generation acetylcholinesterase inhibitor. Huperzine A has a strong inhibitory effect on acetylcholinesterase at low doses, resulting in a significant increase in acetylcholine (Ach) content in the target site.

For rational design for AChE inhibitors based on the hypothesis of dual binding, i.e., binding to the catalytic and peripheral sites, an automated computer docking system design can be used to synthesize tacrine analogs with superior therapeutic potential. This system attempts to maximize the potential for energetically favorable binding sites in the AChE active site gorge by rotating the incoming substrate-tacrine and matching each available conformation of these two counterparts. On the other hand, with this docking system, the existence of the peripheral site near the opening of the enzyme gorge was further confirmed. This is reasonable, because the outer peripheral site may hinder the substrate from entering the lower portion of the enzyme gorge, which in turn lowers the efficacy of hydrolysis. Through structural modification of tacrine, a dimeric tacrine analog linked by an alkylene chain (so named bis(n)-tacrine) offers a much stronger potency and selectivity towards AChE, where bis(n)-tacrine with an appropriate chain length linker (such as heptylene) simultaneously binds at both the catalytic and the peripheral sites.

Alkylene-linked bis(n)-tacrine analogs are obtained by the scheme shown below:

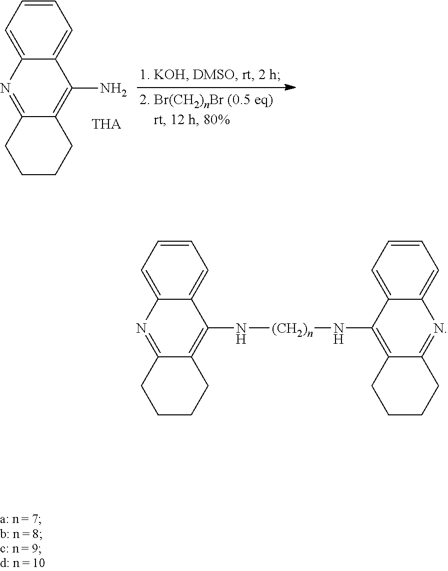

Effective alkylene-bridged analogs require chains of the correct length (number of methylene units) coupled to the parent structure at the appropriate point. The synthesized dimeric compounds were tested in vitro for potency and demonstrated that bis(7)-tacrine (B7C) proved 1,000 times more potent and 10,000 times more selective in inhibiting rat brain AChE as compared with tacrine. The results are shown in Table 1.

TABLE 1

| | Tacrine and | AChE | BuChE | Selectivity for |
|---|---|---|---|---|
| Entry | analogs | IC₅₀(nM) | IC₅₀(nM) | AChE |
| 1 | Tacrine | 590 ± 37 | 44 ± 2.0 | 0.1 |
| 2 | a | 0.40 ± 0.025 | 390 ± 66 | 980 |
| 3 | b | 0.66 ± 0.20 | 340 ± 13 | 520 |
| 4 | c | 0.77 ± 0.11 | 190 ± 30 | 250 |
| 5 | d | 3.1 ± 0.75 | 440 ± 100 | 140 |

For the inhibitory efficacy and selectivity of different length alkylene linker-linked tacrine homodimers against AChE and BChE, see Table 3 in Carlier, Paul R., et a1. “Evaluation of short-tether bis-THA AChE inhibitors. A further test of the dual binding site hypothesis.” Bioorganic & medicinal chemistry 7.2 (1999): 351-357.

In vitro, bis(7)-tacrine is 149-fold more potent and 250-fold more selective for AChE inhibition than tacrine. In vivo (rat cortex or whole brain), 30 minutes after a single dose oral administration, bis(7)-tacrine is 10-fold more potent for AChE inhibition than tacrine. Further, bis(7)-tacrine is less toxic than tacrine and has a significantly improved therapeutic index.

HA inhibits AChE with excellent potency and exquisite selectivity. However, the supply of this natural product is very limited and the complex tricyclic structure makes total synthesis extremely expensive. With the goal of eventually designing potent HA analogs that require less synthetic effort, a series of new dimers, bis(n)-hupyridone (EnE), were synthesized using modified HA molecules. It has been found that these novel dimers exhibit high potency as AChE inhibitors despite the fact that the activity of the hupyridone or similar monomer is extremely low. The anti-acetylcholinesterase activities of the dimers, bis(n)-hypyridone, were evaluated in vitro and in vivo using a spectrophotometric method based on the Ellman method. It was found that the IC₅₀ of bis(12)-hupyridone (E12E) for AChE was estimated to be 52 nM and the selectivity to be 185 (Table 2). E12E was about 4.4 times more potent than tacrine and about twice as potent as HA in inhibiting rat brain AChE.

TABLE 2

| | | | AChE | BuChE | Selectivity for |
|---|---|---|---|---|---|
| Entry | Drug [symbol] | n | IC₅₀ (nM)^{b} | IC₅₀ (nM) [symbol] | AChE^{d} [symbol] |
| 1 | E10E | 10 | 151 ± 36 | 1820 ± 70 | 12.1 |
| 2 | E11E | 11 | 84 ± 5 | 1160 ± 80 | 13.8 |
| 3 | E12E (S, S) | 12 | 52 ± 8 | 9600 ± 300 | 185.0 |
| 4 | E13E | 13 | 52 ± 9 | 16700 ± 650 | 321.0 |
| 5 | E14E | 14 | 240 ± 50 | 59500 ± 10100 | 148.0 |
| 6 | E12E (R, R) | 12 | 3130 ± 790 | 297000 ± 78400 | 94.9 |

[symbol]

indicates data missing or illegible when filed

^{a}Drug E12E (S,S)=(S,S)-(−)-N, N′-di-5′-(5′, 6′7′,8′-tetrahydroquinolin-2-onyl)-1,12-diaminododecane, bis-hydrochloride; ^{b}Assay performed using rat cortex homogenate, in the presence of ethopropazine as a specific BuChE inhibitor; ^{c}Assay using rat serum, in the presence of BW284c51 as a specific AChE inhibitor; and ^{d}Selectivity for AChE is defined as IC₅₀(BuChE)/IC₅₀ (AChE).

Considering that the AChE peripheral site binds a variety of hydrophobic amine ligands, a tacrine heterodimer was synthesized containing an easily synthesized fragment of HA molecule that was simplified by removing both the three-carbon bridge (C₆-C₈) and C₁₁-ethylidene. 5-Amino-5,6,7,8-tetrahydro-2(1H)-quinolinones are known to be extremely weak inhibitor [IC₅₀>100,000 nM]; however, it seemed likely that they could bind effectively to the peripheral site when linked to another high-affinity catalytic site ligand. The synthesis of the desired HA-tacrine hybrid is quite straightforward. In only 4 steps from commercially available starting materials, heterodimers were obtained in 30-50% yield

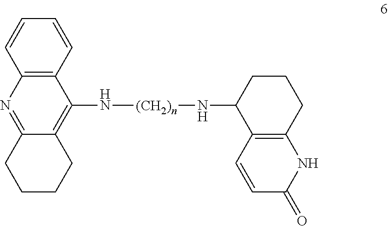

(see, e.g., Pergamon Bioorganic & Medicinal Chemistry Letters 9 (1999) 2335-2338 BIOORGANIC & MEDICINAL CHEMISTRY LETTERS Potent, Easily Synthesized Huperzine A-Tacrine Hybrid Acetylcholinesterase Inhibitors).

As expected, the simplified HA monomer is a extremely weak inhibitor (ICso 500,000 nM). But as hoped, these heterodimers displayed dramatic potency enhancements compared to both this monomer and tacrine. As can be seen, potency is maximized with a linker of ten methylenes (i.e., n=10): HA(10)-tacrine has nanomolar affinity for AChE (IC₅₀≈8.8 nM) and is 13-fold more potent than HA.

Professor Camps from the University of Barcelona reported another excellent tacrine/HA hybrid. Camps directly fused the two structures to give a dimer

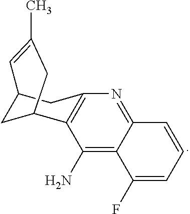 [SMILES: CC1=CC2Cc3nc4cccc(F)c4c(N)c3C(C1)C2]

This dimer is 21-fold more potent than HA, and is thus slightly more potent than HA (10)-tacrine, but HA (10)-tacrine is by far simpler to be synthesized.

The dimer of the present application may also be an HA dimer, bis(n)-hupyridone as shown below, where n=8-16. See GB2360518A for its synthesis method.

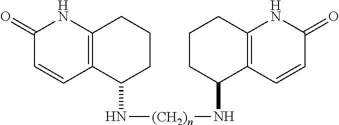

In particular, when n=12, the resulting compound is bis(12)-hupyridone (E12E).

Homo- or heterodimerization of tacrine and huperzine A results in a stronger function compared with traditional acetylcholinesterase inhibitors, since these dimers target not only the enzymatic but also the non-enzymatic function of acetylcholinesterase. Preferably, the dimeric acetylcholinesterase inhibitor described herein is selected from hupyridone (10)-tacrine (A10E), bis(3)-tacrine (B3C), bis(7)-tacrine (B7C) and bis(12)-hupyridone (E12E).

The dimeric acetylcholinesterase inhibitors herein are disease-specific for the treatment or prevention of osteoarthritis, which effectively relieve synovial inflammation and exert chondro- and osteoprotection by targeting acetylcholinesterase in the cholinergic anti-inflammatory pathway. The applicant of this application has found that the dimeric acetylcholinesterase inhibitors of the present application are capable of reducing inflammatory activation of chondrocytes, increasing bone formation and reducing bone resorption. The applicant of this application has also found that the dimeric acetylcholinesterase inhibitors of the present application reduce catabolic factors and increase anabolic factors upon IL-1β-induced chondrocyte activation. Furthermore, since acetylcholinesterase is involved in oxidative stress, acetylcholinesterase inhibitors may also slow senescence.

Without being bound by any theory, it is believed that the disease-modifying effect can be attributed to the influence of the dimeric acetylcholinesterase inhibitors herein on the cholinergic system in the joint, i.e. they will improve bone structure, reduce the inflammatory activation of chondrocytes and reduce oxidative stress-induced senescence, which are three important aspects of osteoarthritis. This can be demonstrated by the observed reduction in inflammatory markers following the treatment with the dimeric acetylcholinesterase inhibitors herein after Il-1β activation of the chondrocytes. Moreover, the present application also demonstrates that the dimeric acetylcholinesterase inhibitors herein are related to the anti-catabolic and/or anabolic effects on bone homeostasis and chondrocytes. Thus, the dimeric acetylcholinesterase inhibitors provided herein can act as disease modifying drugs for osteoarthritis.

Osteoarthritis leads to pain and disability that can only be temporarily relieved by current treatment options, including symptom relief (i.e., pain medication) and health education (i.e. weight control and exercise). Moreover, the lack of treatment to improve the course of the disease leads to complications, the treatment of which also requires high economic costs for patients.

The dimeric acetylcholinesterase inhibitors provided herein more specifically target joint inflammation and alter the progression of the disease, thereby enhancing the treatment of osteoarthritis and improving the quality of life of patients suffering from this disease. Accordingly, the dimeric acetylcholinesterase inhibitors provided in the present application reduce complications associated with osteoarthritis and improve treatment outcomes, leading to a reduction in healthcare costs as well as treatment costs associated with complications.

### Osteoarthritis

Osteoarthritis, the most common form of arthritis, is the most prevalent joint disease, with an estimated 10% of men and 18% of women over 60 years old affected.

Osteoarthritis (OA), a nonspecific joint inflammation characterized by articular cartilage destruction, subchondral osteonecrosis and joint space narrowing, is a progressive chronic disease suggestive of senescence.

The articular cartilage, or “hyaline cartilage”, of healthy vertebrates (including humans and other mammals) is a semi-transparent, opalescent connective tissue characterized by a columnar growth pattern of chondrocytes in an extracellular matrix (ECM) composed predominantly of proteoglycans, type II collagen, and water. Articular cartilage provides an effective weight-bearing cushion to prevent contact between opposing bones in a joint and thus is critical to the normal function of the joint.

Articular cartilage degeneration is the primary concern in osteoarthritis. Homeostasis and integrity of articular cartilage rely on its biochemical and biomechanical interplay with subchondral bone and other joint tissues. Subchondral bone provides the mechanical support for overlying articular cartilage during the movement of joints and undergoes constant adaptation in response to changes in the mechanical environment through modeling or remodeling, in the situation of instability of mechanical loading on weight-bearing joints, such as occurs with ligament injury, excessive body weight, or weakening muscles during aging, the subchondral bone and calcified cartilage zone undergo changes. For instance, rupture of anterior cruciate ligament (ACL) increases the risk of knee osteoarthritis, and approximately 20-35% of individuals with osteoarthritis are estimated to have had an incidental ACL tear. Clinically, osteophyte formation, subchondral bone sclerosis, disruption of tidemark accompanied by angiogenesis at the osteochondral junction, and articular cartilage degeneration are characteristics of osteoarthritis. Bone marrow lesions are closely associated with pain and implicated to predict the severity of cartilage damage in osteoarthritis. In healthy articular cartilage, matrix turnover remains at relatively low rates and chondrocytes resist proliferation and terminal differentiation. During progression of osteoarthritis, type X collagen, alkaline phosphatase, Runt-related transcription factor 2 (RUNX2), and MMP13 are expressed in articular chondrocytes with decreased proteoglycans and expanded calcified cartilage zones in articular cartilage.

Articular cartilage is not only susceptible to damage by joint trauma, but also to a gradual process of erosion. Initially, such an erosion may be simply an asymptomatic “partial thickness defect” in which an area of reduced hyaline cartilage does not penetrate completely to the subchondral bone. The base of a partial thickness defect is usually not painful and is typically only detected during arthroscopic examination. However, if the erosive process is not treated, the base of a partial thickness defect may continue to wear away and the diameter of the defect may increase such that the defect eventually progresses to a “full thickness defect” that penetrates the underlying bone. Such full thickness defects may become sufficiently large that surfaces of opposing bones of the joint make contact and begin to erode one another, leading to inflammation, pain, and other degenerative lesions, i.e., the typical symptoms of osteoarthritis. Osteoarthritis is thus a degenerative, progressive, and crippling disease that results in joint deformity, instability, impairment, and pain.

The most common symptoms of osteoarthritis are joint pain and stiffness. Usually the symptoms progress slowly over years. It may initially occur only after exercise, but becomes constant over time. Other symptoms may include joint swelling, decreased range of motion, and, when the back is affected, weakness or numbness of the arms and legs. The most commonly involved joints are the two near the ends of the fingers and the joint at the base of the thumbs, the knee and hip joints, and the joints of the neck and lower back. Joints on one side of the body are generally more affected than those on the other side. These symptoms can interfere with work and normal daily activities.

Causes of osteoarthritis, in addition to age, genetics, injury, obesity, and hypertension are predisposing factors for the development of osteoarthritis. Risk is greater in those who are overweight, have legs of different lengths, or have jobs that result in high levels of joint stress. Osteoarthritis is believed to be caused by mechanical stress on the joint and low-grade inflammatory processes. It develops as cartilage is lost and the underlying bone becomes affected. As pain may make it difficult to exercise, muscle loss may occur.

Diagnosis of osteoarthritis is typically based on signs and symptoms, with medical imaging and other tests used to support or rule out other problems. In contrast to rheumatoid arthritis, in osteoarthritis the joints do not become hot or red.

Treatments include exercise, decreasing joint stress such as by rest or use of a cane, and pain medications. Weight loss may help in those who are overweight. Pain medications may include paracetamol (acetaminophen) as well as NSAIDs such as naproxen or ibuprofen. If the impact of symptoms of osteoarthritis on quality of life is significant and more conservative management is ineffective, joint replacement surgery or resurfacing may be recommended. An artificial joint typically lasts 10 to 15 years.

Biological joint replacement involves replacing diseased tissue with new tissue. This can come from either a person (autologous transplant) or a donor (allogeneic transplant). People receiving joint transplants (allogenic cartilage transplants) do not need to take immunosuppressants because of the limited immune response of bone and cartilage tissue. Autologous articular cartilage transfer from non-weight-bearing areas to those compromised, called the osteochongdral autograft transfer system (OATS), is one possible procedure under investigation. Autologous chondrocyte implantation is also an option when the missing cartilage is a local defect.

### Therapeutic Methods and Uses

The present application provides a method for preventing or treating an osteoarticular disease in a patient in need thereof, comprising administering to the patient a therapeutically effective amount of an acetylcholinesterase inhibitor.

The present application also relates to the use of an acetylcholinesterase inhibitor in the prevention or treatment of an osteoarticular disease and in one or more of: providing osteoprotection; improving bone structure; maintaining bone homeostasis; reducing inflammatory activation of chondrocytes; providing an anti-catabolic and/or pro-anabolic effect on chondrocytes; reducing telomere length; and reducing oxidative stress-induced senescence.

The present application also relates to the use of an acetylcholinesterase inhibitor for the preparation of a drug for the prevention or treatment of an osteoarticular disease; and the use of an acetylcholinesterase inhibitor in the preparation of a drug for one or more of: providing osteoprotection; improving bone structure; maintaining bone homeostasis; reducing inflammatory activation of chondrocytes; providing an anti-catabolic and/or pro-anabolic effect on chondrocytes; reducing telomere length; and reducing oxidative stress-induced senescence.

In some embodiments, the osteoarticular disease includes osteoarthritis, rheumatic arthritis, rheumatoid arthritis, bursitis, synovitis, cervical spondylosis, lumbar spondylosis, scapulohumeral periarthritis, hyperosteogeny, ligament injury and local joint inflammation. Preferably, the osteoarticular disease is osteoarthritis.

In some embodiments, the acetylcholinesterase inhibitor is selected from donepezil, 7-methoxytacrine, huperzine A, galantamine, ambenonium chloride, and the like.

In some embodiments, the dimeric acetylcholinesterase inhibitor is a tacrine homodimer, i.e., bis(n)-tacrine (also known as bis(n)-Cognex, abbreviated as B(n)C), as shown below:

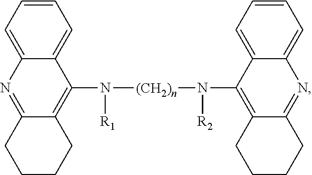

wherein R₁ and R₂ are each independently H or C₁₋₄alkyl, and n=2-10.

Preferably, R₁ and R₂ are both H.

More preferably, the tacrine homodimer is B(3)C or B(7)C:

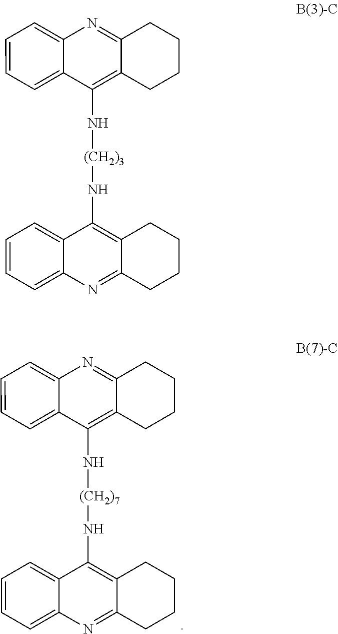

In some embodiments, the dimeric acetylcholinesterase inhibitor is a huperzine A (HA)-tacrine heterodimer as shown below:

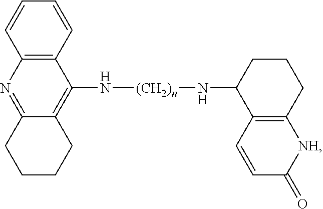

wherein n=4-12.

Preferably, n=10, i.e., the dimer is a huperzine A (HA)-tacrine heterodimer (abbreviated as A10E) as shown below:

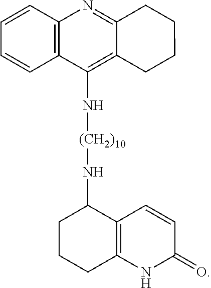

In some embodiments, the dimeric acetylcholinesterase inhibitor is a huperzine A (HA) homodimer having the formula:

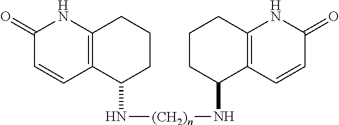

wherein n=10-14.

Preferably, n=12, i.e., the dimer is bis(12)-hupyridone (E12E) as shown below:

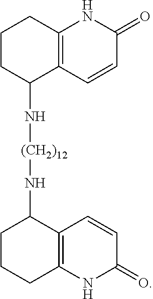

The term “effective” as used herein means adequate to accomplish a desired, expected, or intended result. More specifically, a “therapeutically effective amount” provided herein refers to an amount of an acetylcholine inhibitor of the invention, alone or in combination with another therapeutic agent (e.g., a TGF-β inhibitor and/or a different other therapeutic agent) necessary to provide the desired therapeutic effect, e.g., an amount effective to prevent, alleviate or ameliorate symptoms of a disease or prolong the survival of the subject being treated. In a specific embodiment, the term “therapeutically effective amount” provided herein refers to an amount of an acetylcholine inhibitor necessary to provide the desired therapeutic effect, e.g., an amount effective to prevent, alleviate or ameliorate symptoms of a disease or condition or prolong the survival of the subject being treated. In a more specific embodiment, a therapeutically effective amount of an AChE inhibitor refers to an amount necessary to treat or prevent osteoarthritis, prevent onset of ligament injury-induced osteoarthritis, prevent onset of osteoarthritis in an unstable joint, or reduce the degeneration of articular cartilage in a joint.

As would be appreciated by one of ordinary skill in the art, the exact amount required will vary from subject to subject, depending on age, general condition of the subject, the severity of the condition being treated, the specific compound and/or composition administered, and the like. An appropriate “therapeutically effective amount” in any individual case can be determined by one of ordinary skill in the art by reference to the pertinent texts and literature and/or by using routine experimentation.

As used herein, the terms “treatment”, “treating”, “treat” and the like refer to obtaining a desired pharmacologic and/or physiologic effect. The effect may be prophylactic in terms of completely or partially preventing a disease, condition or symptom thereof and/or may be therapeutic in terms of a partial or complete cure for a disease or condition and/or adverse effect attributable to the disease or condition. “Treatment” as used herein covers any treatment of a disease or condition in a subject, particularly a human, and includes: (a) preventing the disease or condition from occurring in a subject, which may be predisposed to the disease but has not yet been diagnosed with it; (b) inhibiting the disease or condition, i.e., arresting its development; and (c) relieving the disease or condition, e.g., causing regression of the disease or condition, e.g., to completely or partially eliminate a symptom of the disease or condition.

### Pharmaceutical Compositions

The pharmaceutical compositions of the present application may comprise an effective amount of an acetylcholinesterase inhibitor as well as a pharmaceutically acceptable excipient. As noted above, the term “effective” as used herein means adequate to accomplish a desired, expected, or intended result. More specifically, an “effective amount” or a “therapeutically effective amount” is used interchangeably and refers to an amount of at least one acetylcholinesterase inhibitor, perhaps in further combination with, yet another therapeutic agent, necessary to provide the desired treatment or therapeutic effect, e.g., an amount that is effective to prevent, alleviate, treat or ameliorate symptoms of a disease or condition or prolong the survival of the subject being treated. In a particular embodiment, the pharmaceutical compositions of the present application are administered in a therapeutically effective amount to treat patients having osteoarticular diseases, in particular osteoarthritis, or patients at risk of developing osteoarthritis including patients suffering from a ligament injury.

As would be appreciated by one of ordinary skill in the art, the exact dosage required for acetylcholinesterase inhibitors will vary from subject to subject, depending on age, general condition of the subject, the severity of the condition being treated, the specific compound and/or composition administered, and the like. An appropriate “therapeutically effective amount” in any individual case can be determined by one of ordinary skill in the art by reference to the pertinent texts and literature and/or by using routine experimentation.

The pharmaceutical compositions of the present application are in biologically compatible form suitable for administration in vivo for subjects. The pharmaceutical compositions further comprise a pharmaceutically acceptable carrier. The term “pharmaceutically acceptable” means approved by a regulatory agency of the Federal or a state government or listed in the U.S. Pharmacopeia or other generally recognized pharmacopeia, for use in animals, and more particularly in humans. The term “carrier” refers to a diluent, adjuvant, excipient, or vehicle with which the acetylcholinesterase inhibitor is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, rice, flour, chalk, silica gel, sodium stearate, glycerol monostearate, talc, sodium chloride, dried skim milk, glycerol, ethylene glycol, water, ethanol and the like. The pharmaceutical composition may also contain minor amounts of wetting or emulsifying agents, or pH buffering agents.

The pharmaceutical compositions of the present invention can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of an acetylcholinesterase inhibitor together with a suitable amount of a pharmaceutically acceptable carrier so as to provide the form for proper administration to the patient. The formulation should suit the mode of administration.

The pharmaceutical compositions of the present invention may be administered by any particular ecific route of administration, including but not limited to oral, parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. The most suitable route is oral administration or injection. In certain embodiments, an injection into the affected joint area is preferred.

In general, the pharmaceutical compositions comprising a acetylcholinesterase inhibitor may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated: the route of administration; the renal and hepatic function of the patient; and the specific pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., pharmaceutical compositions comprising at least one acetylcholinase inhibitor (and optionally in combination with another therapeutic agent)) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosage of a pharmaceutical composition disclosed herein may be adjusted when used in combination to achieve desired effects. On the other hand, the dosages of the pharmaceutical composition and various therapeutic agents may be independently optimized and combined to achieve a synergistic result wherein the pathology is reduced more than it would be if either was used alone.

In the case of injections, it is usually convenient to administer in an amount of about 1-30 mg, about 5-25 mg or about 10-20 mg per day to adults (please modify as appropriate). Preferably, about 3 mg, 5 mg, 8 mg or 12 mg per day is administered to adults (modify as appropriate). In the case of other animals, the dose calculated for 60 kg may be administered as well.

As a non-limiting example, treatment of patients can be on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the pharmaceutical compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days.

Alternatively, the pharmaceutical compositions of the present invention may be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions of the present invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively, the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

The pharmaceutical compositions may further be combined with one or more additional therapeutic agents. The determination of the identity and amount of the acetylcholinesterase inhibitors or dimeric acetylcholinesterase inhibitors for use in the pharmaceutical compositions of the present application can be readily made by ordinarily skilled medical practitioners using standard techniques known in the art. In other specific embodiments, the acetylcholinesterase inhibitor can be administered in combination with an effective amount of another osteoarthritis therapeutic agent, e.g., a TGF-beta inhibitor, an IL-1 inhibitor, a corticosteroid, hyaluronic acid, and the like.

Without further elaboration, it is believed that one skilled in the art can, using the preceding description, utilize the present invention to its fullest extent. The examples provided below are illustrative only, and should not be construed as limiting the remainder of the disclosure of the present application in any way whatsoever.

### EXAMPLES

Four dimers were selected based on their superior acetylcholinesterase inhibitory activity, see Table 3 (Journal of Psychopharmacology 14(3) (2000) 275-279; Curr Ah [symbol] heimer Res. 2007) 4, 386-396.).

TABLE 3
Anti-AChE activities of known AChE inhibitors and novel dimers

| | Relative potency | AChE inhibitory |
|---|---|---|
| Drug | (fold change) | IC₅₀ (nM) |
| Tacrine | 1 | 223 |
| Huperzine A | 2 | 114 |
| Donepezil | 33 | 6.7 |
| Rivastigmine | 52 | 4.3 |
| Bis(12)-hupyridone (E12E) | 4 | 52 |
| Tacrine (10)-hupyridone (A10E) | 25 | 8.8 |
| Bis(3)-Cognitin (B3C) | 1 | 254 |
| Bis(7)-Cognitin (B7C) | 149 | 1.5 |

Note:
The relative potency was obtained by dividing the AChE inhibitory IC₅₀ of tacrine with that of each AChE inhibitor.

Methods of synthesizing HA dimers can be found in GB2360518A; HK1042291; U.S. Pat. No. 6,472,408B1; and Carlier, Paul R., et a1. “Dimerization of an inactive fragment of huperzine A produces a drug with twice the potency of the natural product.” Angewandte Chemie 112.10 (2000): 1845-1847; methods for synthesizing tacrine homodimer can be found in Li, W. M., et a1. “East meets West in the search for Alzheimer's therapeutics-novel dimeric inhibitors from tacrine and huperzine A.” Current Alzheimer Research 4.4 (2007): 386-396; and for methods of synthesis of tacrine-HA heterodimers, see Carlier, Paul R., et a1. “Potent, easily synthesized huperzine A-tacrine hybrid acetylcholinesterase inhibitors.” Bioorganic & medicinal chemistry letters 9.16 (1999): 2335-2338.

The following examples illustrate a new application of the above four dimeric acetylcholinesterase inhibitors, i.e., bis(12)-hupyridone (E12E), tacrine(10)-hupyridone (A10E), bis(3)-Cognitin (B3C) and bis(7)-Cognitin (B7C), i.e., for osteoarthritis treatment and prevention. These dimeric acetylcholinesterase inhibitors proved to be capable of acting as disease-modifying drugs in OA treatment, all acting to inhibit disease progression in different osteoarthritis development models.

### Example 1

In this rat model, spontaneously hypertensive rats (SHR) were used to study osteoarthritis, while Wister Kyoto (WKY) rats were used as controls. Donepezil, a commercially available acetylcholinesterase inhibitor, was administered to SHR and WKY rats via abdominal injection at a dose of 2 mg/kg body weight/day for one month. The femurs of SHR and WKY rats were then decalcified and paraffin embedded and sectioned to a thickness of 5 μm.

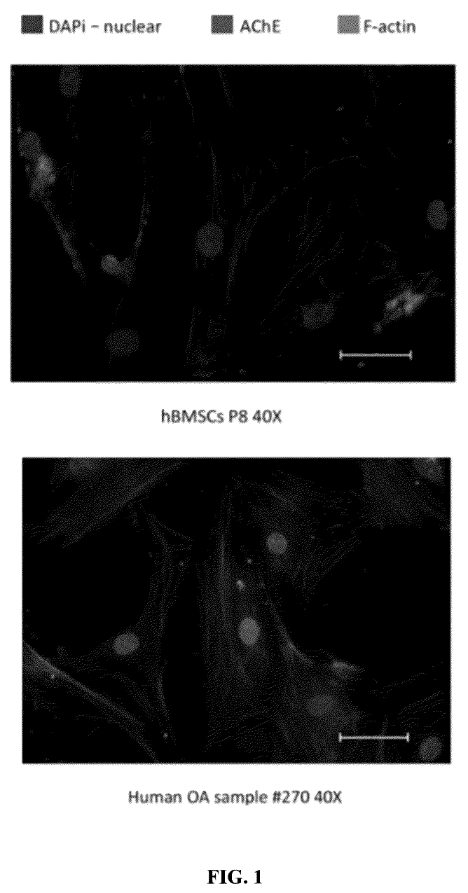

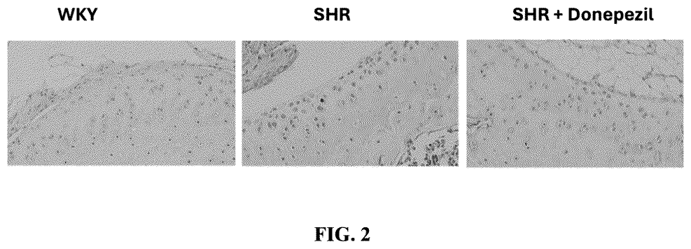

The DAB staining of the rat joint sections for acetylcholinesterase (FIG. 2) showed an increased expression of acetylcholinesterase in osteoarthritic SHR rats compared to the healthy WKY rats. This confirms the previous results shown in FIG. 1. Additionally, after treating the osteoarthritic rats with donepezil, a decrease in acetylcholinesterase expression could be observed, which improves the disease (FIG. 2).

### Example 2

Interleukin 1 beta (IL-1β), a cytokine expressed in OA joints, has been implicated in the pathogenesis of OA. It stimulates the production of NOS (nitric oxide synthase) and Cox-1 (cyclooxygenase 1) and finally leads to a metabolic shift from cells in a resting state to a highly metabolic state associated with an increased production of inflammatory and catabolic factors and a decreased production of anabolic factors.

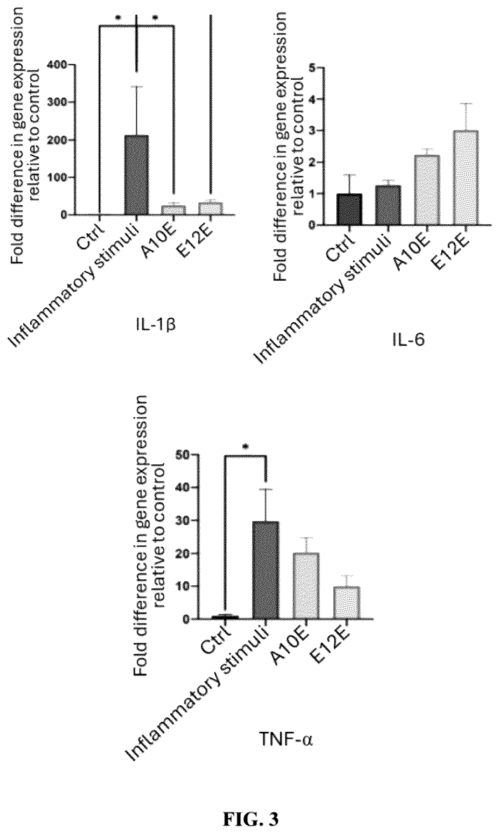

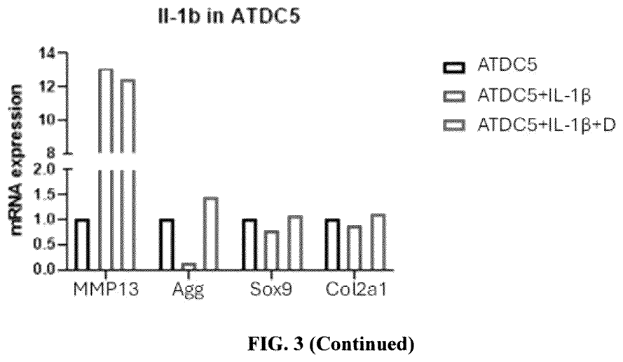

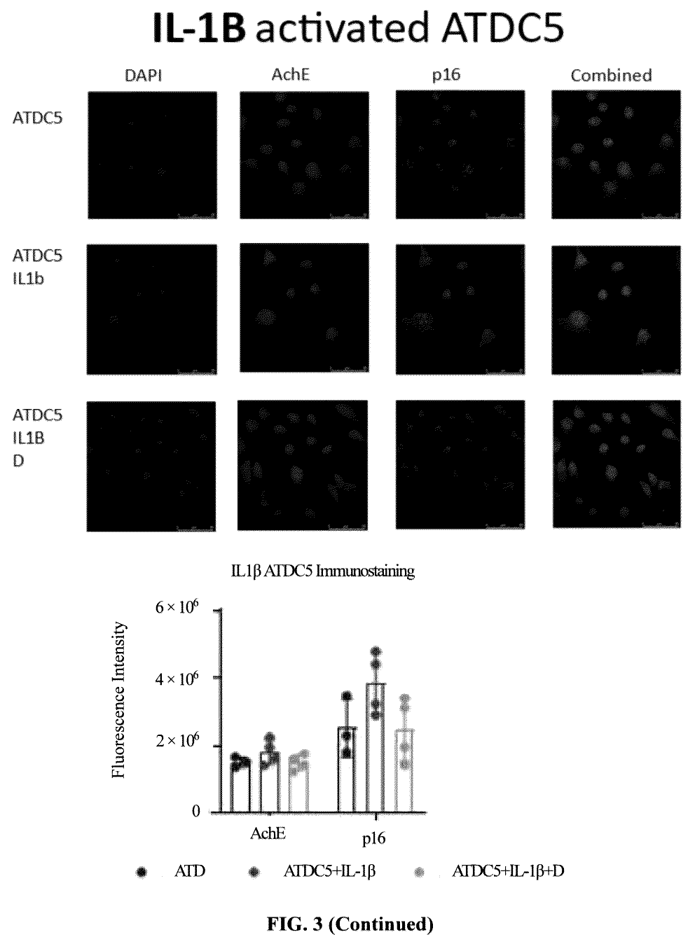

The expression of acetylcholinesterase was increased in IL-1β stimulated chondrocytes (FIG. 3), indicating a role for acetylcholinesterase in OA.

10 ng/ml of exogenous IL-1β was added to 1*10⁶ ATDC5 cells (derived from mouse teratoma cells and characterized as a cholinergic cell line that underwent a sequential process similar to chondrocyte differentiation) in each well of a 6-well plate and incubated for 24 h. An increase in the inflammatory factors IL-1β, TNFα, IL-6 and catabolic factor MMP13 (matrix metalloproteinase 13) was observed by qPCR, while the anabolic factors Agg (aggrecan), Sox9, Col2 decreased (FIG. 3).

The data indicated that the novel dimeric acetylcholinesterase inhibitors of the present application could protect chondrocytes from inflammatory stimuli (IL-1β) by down-regulating key pro-inflammatory cytokines including TNF-alpha (FIG. 3A, where A10E and E12E are dimeric acetylcholinesterase inhibitors).

Additionally, the commercially available acetylcholinesterase inhibitor, donepezil, increased the anabolic and decreased the catabolic factors (FIG. 3B). Finally, 11-10 is also associated with an increase in senescence shown by upregulation of p16. In this study, p16 expression also decreased after treatment with the acetylcholinesterase inhibitor, donepezil (FIG. 3C). D=Donepezil; Il-1b is IL-1 beta; MMP13 is matrix metalloproteinase 13; Agg is Aggrecan; Col2a1 is collagen 2a1.

### Example 3

Hydrogen peroxide is used to induce oxidative stress associated with cellular senescence, which also plays an important role in the development of osteoarthritis.

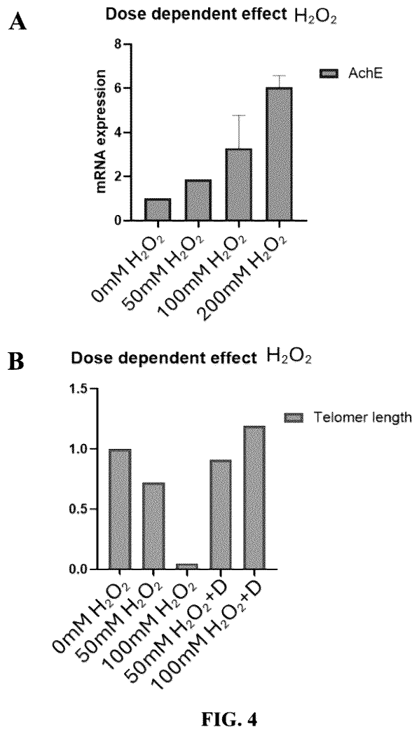

In one experiment, 0 mM, 50 mM, 100 mM and 200 mM H₂O₂ were added to 1*10⁶ ATDC5 cells in different wells in a 6-well plate and the mRNA expression of AChE was measured after 24 h of incubation (FIG. 4A).

In another experiment, 0 mM, 50 mM, 100 mM H₂02, 50 mM H₂O₂+1 μM donepezil (D), and 100 mM H₂O₂+1 μM donepezil (D) were added to 1*10⁶ ATDC5 cells in different wells in a 6-well plate and the telomere length was monitored after 24 h of incubation (FIG. 4B).

The data indicated that increasing concentrations of hydrogen peroxide increased the mRNA expression of acetylcholinesterase in ATDC5 cells (FIG. 4A); meanwhile, acetylcholinesterase inhibitors could prevent telomere length shortening, thereby inhibiting the development of the disease (FIG. 4B).

### Example 4

In addition to cartilage degeneration, subchondral bone loss and microstructural deterioration are also key features of OA. In early OA, an increased bone turnover and structural deterioration is observed and as the disease progresses it becomes sclerotic presenting an increased density and low mineralization. Activation of the cholinergic system holds a good promise to increase osteoblast proliferation and osteoclast apoptosis in order to restore the balance of bone remodeling.

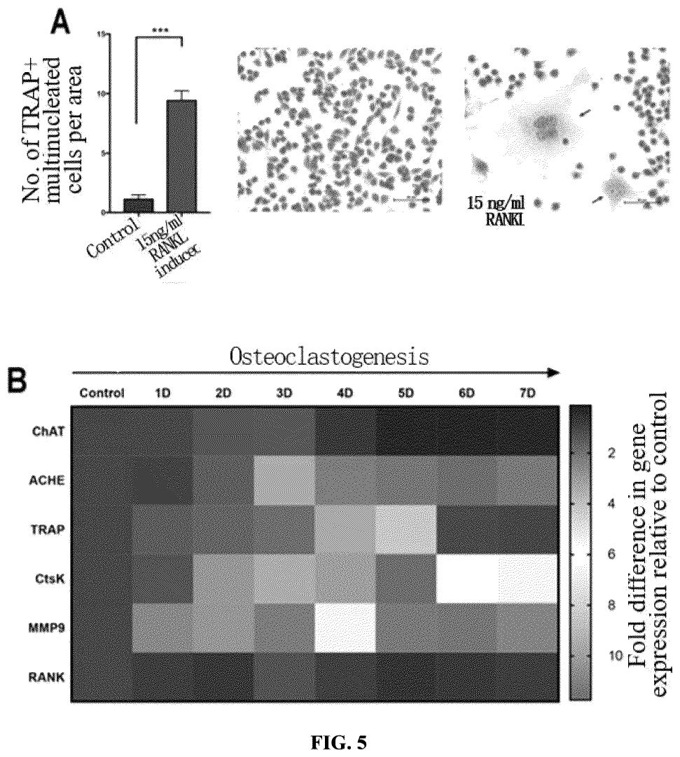

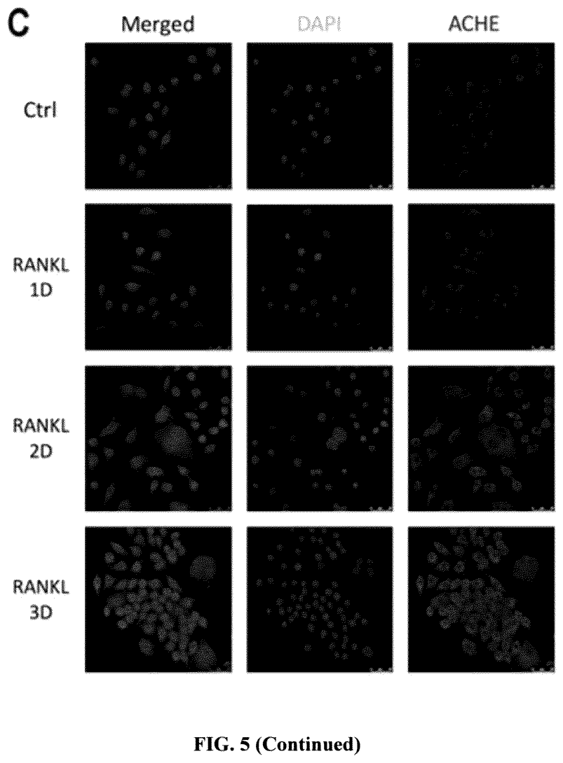

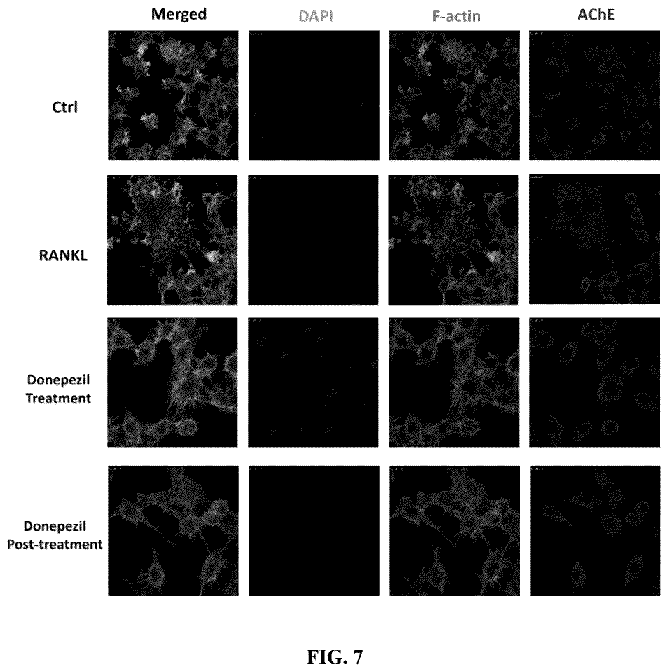

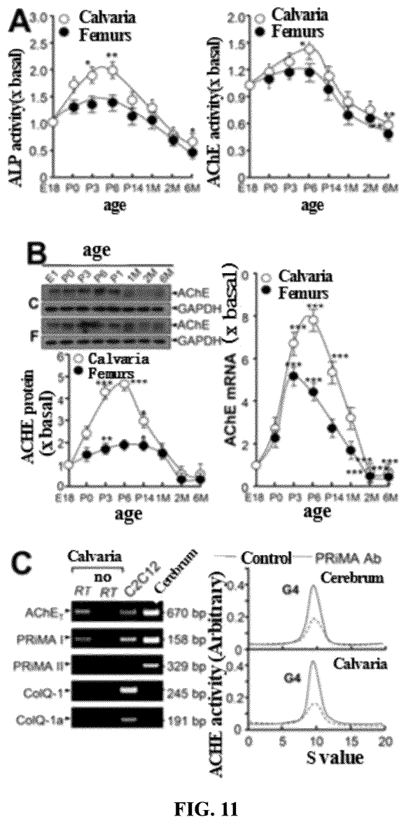

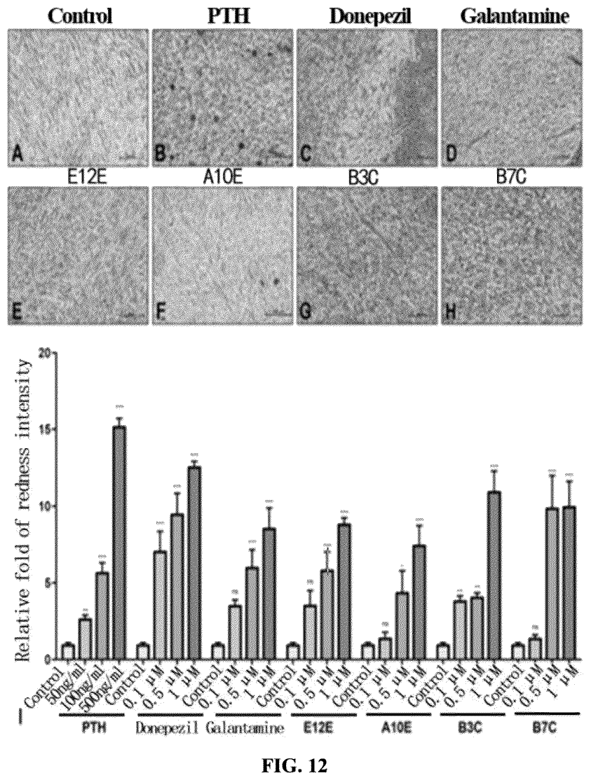

Acetylcholinesterase increases osteoclastogenesis and facilities macrophage fusion via its non-enzymatic function (FIGS. 5-9). Dual blockade of acetylcholinesterase enzymatic and non-enzymatic functional sites could inhibit osteoclast formation and mitigate bone loss both in vitro and in vivo (FIGS. 7-10). Acetylcholinesterase also plays a part in osteoblast differentiation, in particular in mineralization process. Inhibition of acetylcholinesterase can promote osteoblast differentiation and mineralization (FIGS. 11-12).

FIG. 5: RAW 264.7 cells (1*10⁶/well in a 6-well plate) were induced with RANKL (15 ng/ml) for 3-7 days. A) RANKL induced differentiation of RAW 264.7 cells towards multinucleated osteoclasts in vitro; B) AChE mRNA expression levels increased along with other osteoclast differentiation mRNA markers (TRAP, Ctsk, MMP9, RNAK); C) Immunofluorescence images showed that AChE protein levels also increased during osteoclastogenesis. These results indicated that AChE expression increased with osteoclastogenesis.

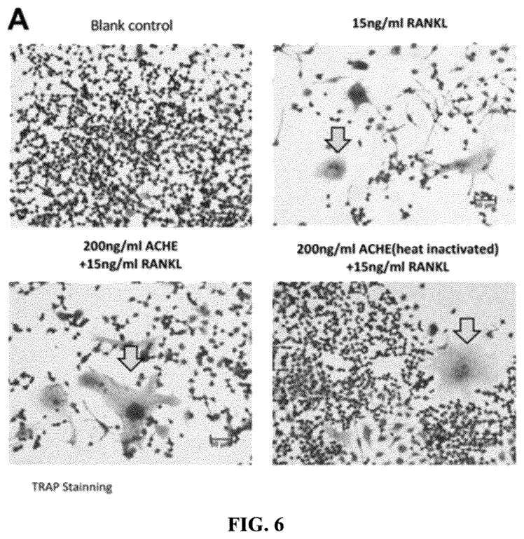

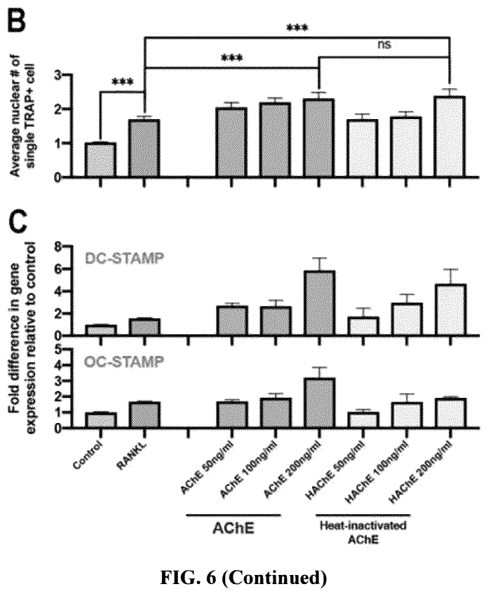

FIG. 6: Similar to the experiment in FIG. 5, RAW 264.7 cells (1*10⁶/well in a 6-well plate) were induced with RANKL (15 ng/ml) for 3 days, except that different concentrations of AChE or heat-inactivated AChE (HAChE) and RANKL were added to the cell culture medium simultaneously. This figure shows that intact or heat-inactivated recombinant mouse AChE invariably promoted macrophage fusion and osteoclastogenesis. A) Both intact and heat-inactivated recombinant mouse AChE stimulated cell-cell fusion and enlargement after RANKL-induced osteoclastogenesis. B) There was no significant difference between intact and heat-inactivated recombinant mouse AChE (HAChE) in terms of the average nuclear number in single TARP+cell. C) The effects of AChE and heat-inactivated recombinant mouse AChE (HAChE) on cell-cell fusion were associated with an increment of cell fusion proteins, DC-STAMP and OC-STAMP.

FIG. 7: Different treatments were performed on RAW 264.7 cells cultured in a 6-well plate at a cell density of 1*10⁶/well. Control group: no treatment; RANKL group: 15 ng/ml RANKL induces osteoclastic differentiation for 4 days; donepezil pretreatment group: donepezil 1 μM pre-treatment for 2 days, followed by 15 ng/ml RANKL osteoclastic differentiation induction for 2 days; donepezil post-treatment group: 15 ng/ml RANKL induces osteoclastic differentiation for 2 days, followed by donepezil 1 μM treatment for 2 days. The figure shows that donepezil inhibited cell-cell fusion of osteoclast precursors.

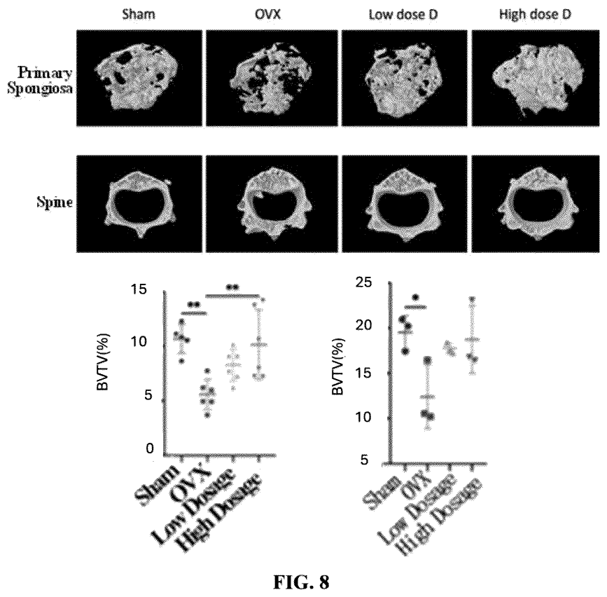

FIG. 8: Twelve 3-month-old Balb/C female mice were randomly divided into four groups. Sham group: The tegument of the back near the ovary was incised and sutured (4 weeks), and normal saline was injected abdominally (4 weeks); OVX control group: bilateral ovariectomy (4 weeks of modelling), abdominal injection of normal saline (4 weeks); low dose Donepezil (D) treatment group: Bilateral ovariectomy (4 weeks of modelling), abdominal injection of donepezil 0.2 mg/kg body weight/day (4 weeks); high dose Donepezil (D) treatment group: bilateral ovariectomy (4 weeks of modelling), abdominal injection of donepezil 2 mg/kg body weight/day (4 weeks). All animals were sacrificed at 2 months after OVX for mirco-CT and histological examination. The mirco-CT images of primary spongiosa of tibia and vertebral body of lumbar spine showed that donepezil exhibited dose-dependent osteoprotection, rescuing OVX (ovariectomy)-induced bone loss.

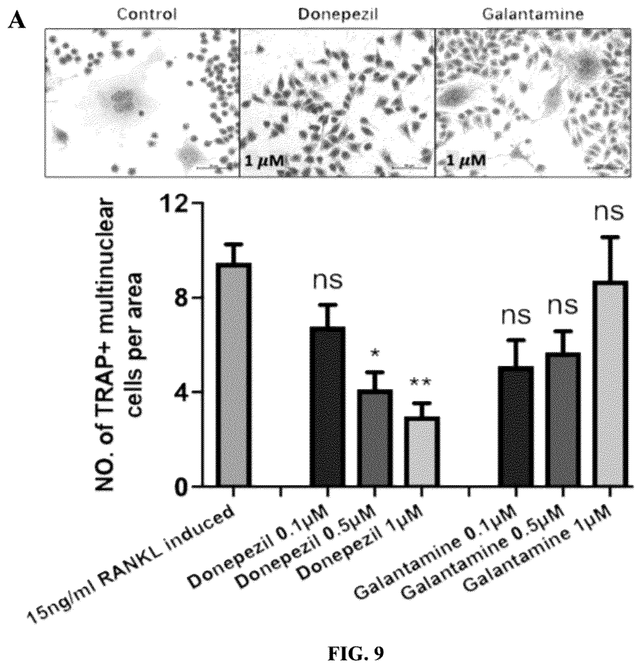

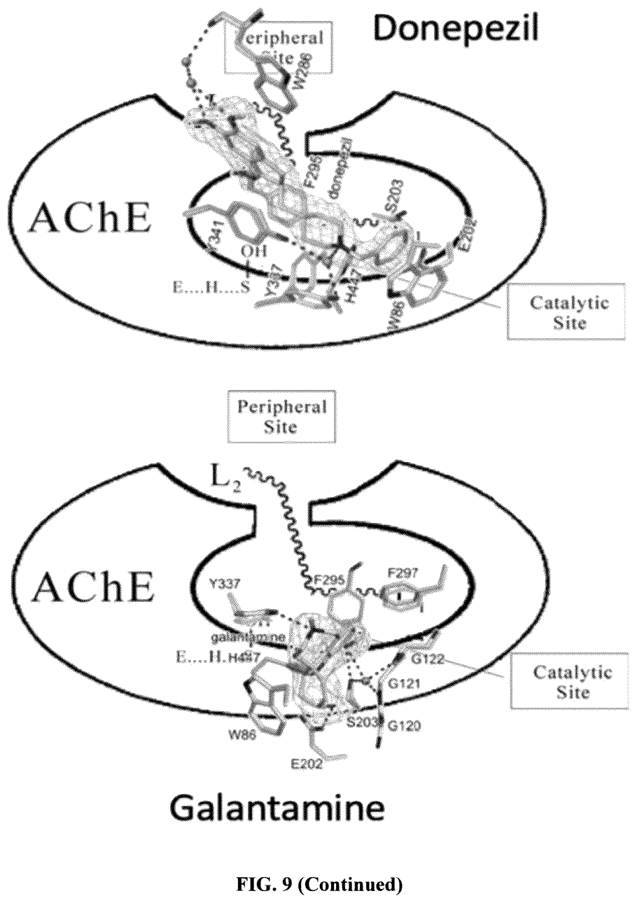

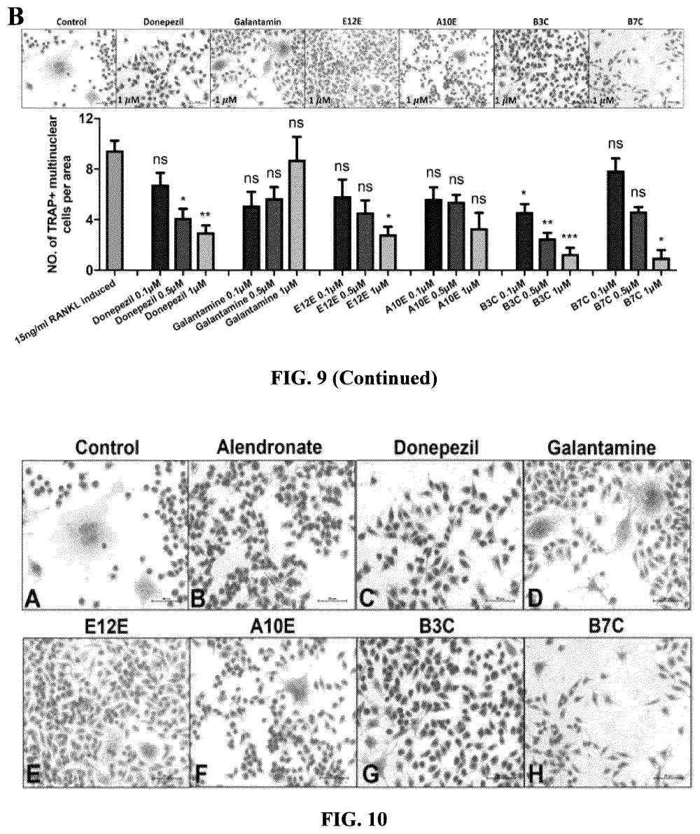

FIG. 9: Similar to the experiment in FIG. 5, RAW 264.7 cells (1*10⁶/well in a 6-well plate) were induced with RANKL (15 ng/ml) for 3 days, except that three concentrations (0.1 μM, 0.5 μM and 1 μM) of donepezil, galantamine and four dimers (E12E, A10E, B3C, B7C) as well as RANKL were added to the cell culture medium simultaneously. FIGS. 9A-9B show that donepezil and four dimers, rather galantamine, suppressed osteoclastogenesis in a dose-dependent manner. Such discrepancy may arise from their different binding patterns in 3D structure of AChE protein. Donepezil and the four dimers occupy the entire binding pocket of AChE whereas galantamine selectively binds only to the catalytic site of AChE.

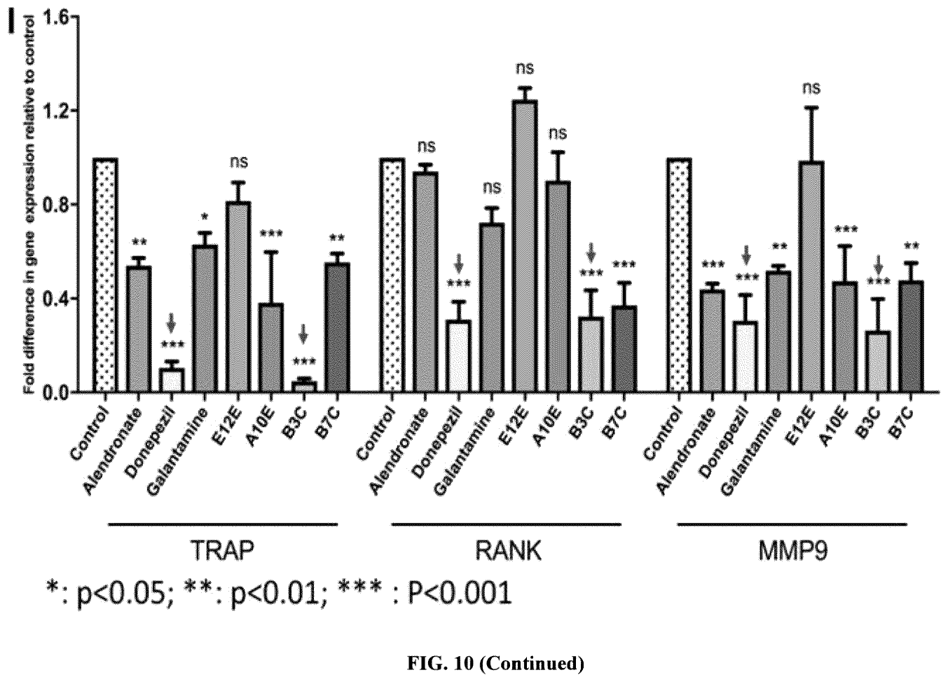

FIG. 10: Experimental conditions were as in FIG. 9 and relative gene expression was measured by qPCR. A-H) showed the inhibitory effects of donepezil, galantamine and dimer inhibitors (all at 1 μM) on osteoclastogenic differentiation of RAW 264.7 cells. This inhibitory effect is particularly pronounced for B3C. I) The comparisons of anti-catabolic effects on osteoclastogenesis among FDA-approved drugs, alendronate, donepezil, galantamine and dimers herein (all at 1 μM). In terms of the mRNA expression of MMP9, RANK, TRAP, the markers for osteoclasts differentiation and maturation, donepezil and the dimeric AChE inhibitors of the present application e.g., B3C are comparable and superior to alendronate.

FIG. 11: Increase of AChE expression and activity during bone development. A) Calvarias and femurs from rats at different stages as indicated were isolated and assayed for ALP and AChE activities. Assay method for ALP activity: With p-nitrophenol phosphate as the substrate and 2-amino-2-methyl-1-propanol or diethanolamine as the acceptor of the phosphate acyl group, under an alkaline environment, ALP catalyzed hydrolysis of 4-NPP to produce free p-nitrophenol, which turned yellow in alkaline solution, and ALP activity units were calculated from the rate of increase in absorbance at 405 nm. Assay method for AChE activity: AChE activity was determined according to Ellman's principle, in which acetylcholinesterase hydrolyzed acetylcholine to generate choline and acetic acid, and choline reacted with sulfhydryl chromogenic agent to generate TNB yellow compound, which was analyzed colorimetrically at 412 nm, reflecting the acetylcholinesterase activity based on the amount of hydrolysate. B) Upper left panel: protein lysates from bone tissues were analyzed by Western blotting; AChE (˜68 kDa) and GAPDH (˜35 kDa) were shown; bottom left panel: quantitation of AChE protein, calibrated from the blots by densitometry; right panel shows the real-time PCR of AChE mRNA, values are expressed as fold of increase to basal reading. C) Left panel: total RNAs were extracted from calvarias and femurs to perform PCR in order to determine the presence of AChE_{T}, PRiMA I, PRiMA II, ColQ-1 and ColQ-1a by using specific primers. PCR products were resolved on a 1% SYBR safe stained agarose gel and visualized under UV light. The identity of the PCR product was confirmed by DNA sequencing. Rat cerebrum and mouse C2C12 RNA served as positive controls. Right panel, molecular forms of AChE in calvarias and femurs were determined by sucrose density gradient analysis. PRiMA-linked G4 AChE was immunodepleted by anti-PRiMA antibodies. Cerebrum served as a positive control for G4 AChE. Enzyme activities are expressed in arbitrary units. Values are in means±S.E., n=4, *, p<0.05; **, p <0.01; ***,p<0.001.

FIG. 12: 1*10⁶/well of murine mesenchymal stem cells (mMSCs) in a 6-well plate were treated with donepezil, galantamine and four dimers (E12E, A10E, B3C and B7C) at three concentrations (0.1 μM, 0.5 μM, 1 μM) and the first-line osteogenic drug PTH and stained with Alizarin Red S. All AChE inhibitors were found to demonstrate dose-dependent anti-anabolic effects at 1 μM concentration, wherein E12E and A10E both promoted bone mineralization, with their promotion strength positively correlated with drug concentration; the promotive effects of donepezil and bis(3)-tacrine (B3C) were observed at the concentration of 1 μM.

It should be understood that the detailed examples and embodiments described herein are given by way of example, for illustration only, and are not to be construed as limiting of the invention in any way. Various modifications or changes in light thereof will be taught to persons skilled in the art and such modifications and changes are included within the spirit and purview of this application and are considered within the scope of the appended claims. For example, the relative quantities of the ingredients may be varied to optimize the desired effects, additional ingredients may be added, and/or similar ingredients substituted for one or more of the ingredients described. Additional advantageous features and functionalities associated with the methods and uses of the disclosure will be apparent from the appended claims. In addition, Those skilled in the art will recognize, or be able to ascertain using no more than routine experimentation, many equivalents to the specific embodiments described herein. Such equivalents are intended to be encompassed by the claims.

### REFERENCES

Li, Wenming, et a1. “Novel anti-Alzheimer's dimer bis(7)-cognitin: cellular and molecular mechanisms of neuroprotection through multiple targets.” Neurotherapeutics 6.1 (2009): 187-201.
Sussman, Joel L., et a1. “Atomic structure of acetylcholinesterase from Torpedo californica: a prototypic acetylcholine-binding protein.” Science 253.5022 (1991): 872-879.
Pergamon Bioorganic & Medicinal Chemistry Letters 9 (1999) 2335-2338 BIOORGANIC & MEDICINAL CHEMISTRY LETTERS Potent, Easily Synthesized Huperzine A-Tacrine Hybrid Acetylcholinesterase Inhibitors.
Carlier, Paul R., et a1. “Dimerization of an inactive fragment of huperzine A produces a drug with twice the potency of the natural product.” Angewandte Chemie 112.10 (2000): 1845-1847.
East Meets West in the Search for Alzheimer's Therapeutics—Novel Dimeric Inhibitors from Tarcine and Huperzine A. Curr Ahzheimer Res. (2007) 4, 386-396.
Carlier, Paul R., et a1. “Potent, easily synthesized huperzine A-tacrine hybrid acetylcholinesterase inhibitors.” Bioorganic & medicinal chemistry letters 9.16 (1999): 2335-2338.
Acetylcholinesterase Inhibitors: Pharmacology and Toxicology. Curr Neuropharmacology, 2013, 11, 315-335.
   GB2360518A
   HK1042291
   U.S. Pat. No. 6,472,408B1
   CN200810142156.8

All publications cited herein, including all journal articles, books, manuals, published patent applications, and issued patents, are hereby incorporated by reference. In addition, the meanings of some terms and phrases employed in the specification, examples, and appended claims are provided. The definitions are not meant to be limiting in nature and serve to provide a clearer understanding of certain aspects of the present invention.

1-16. (canceled)

17. A method of preventing or treating an osteoarticular disease in a subject in need thereof, the method comprising administering a therapeutically effective amount of an acetylcholinesterase inhibitor to the subject.

18. The method according to claim 17, wherein the osteoarticular disease is selected from the group consisting of osteoarthritis, rheumatic arthritis, rheumatoid arthritis, bursitis, synovitis, cervical spondylosis, lumbar spondylosis, scapulohumeral periarthritis, hyperosteogeny, ligament injury, and local joint inflammation.

19. The method according to claim 17, wherein the acetylcholinesterase inhibitor is selected from the group consisting of: 7-methoxytacrine, huperzine A, donepezil, galantamine and ambenonium chloride.

20. The method according to claim 17, wherein the acetylcholinesterase inhibitor is a dimeric acetylcholinesterase inhibitor.

21. The method according to claim 20, wherein the dimeric acetylcholinesterase inhibitor has the structure of formula A-L-B, wherein A and B are acetylcholinesterase inhibitor monomers and may be the same or different, L is an optional linker and, when present, is an alkylene —(CH₂)n—, wherein n is an integer from 1 to 20.

22. The method according to claim 21, wherein A and B are independently selected from tacrine and huperzine A.

23. The method according to claim 20, wherein the dimeric acetylcholinesterase inhibitor is a tacrine homodimer, i.e., bis(n)-tacrine, as shown below: 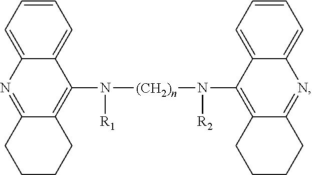
wherein R₁ and R₂ are each independently H or C₄alkyl, and n=2-10.

24. The method according to claim 22, wherein the dimeric acetylcholinesterase inhibitor is a huperzine A-tacrine heterodimer, i.e., huperzine A-(n)-tacrine, as shown below: 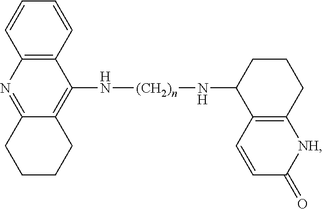
wherein n=4-12.

25. The method according to claim 22, wherein the dimeric acetylcholinesterase inhibitor is a huperzine A homodimer as shown below: 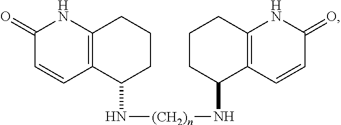
wherein n=10-14.

26. The method according to claim 20, wherein the dimeric acetylcholinesterase inhibitor is

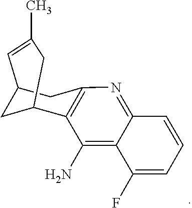 [SMILES: CC1=CC2Cc3nc4cccc(F)c4c(N)c3C(C1)C2]

27. A pharmaceutical composition for preventing or treating an osteoarticular disease comprising a therapeutically effective amount of an acetylcholinesterase inhibitor and a pharmaceutically acceptable carrier.

28. The pharmaceutical composition according to claim 27, wherein the osteoarticular disease is selected from the group consisting of osteoarthritis, rheumatic arthritis, rheumatoid arthritis, bursitis, synovitis, cervical spondylosis, lumbar spondylosis, scapulohumeral periarthritis, hyperosteogeny, ligament injury and local joint inflammation.

29. The pharmaceutical composition according to claim 27, wherein the acetylcholinesterase inhibitor is selected from the group consisting of: tacrine, huperzine A, donepezil, galantamine and ambenonium chloride.

30. The pharmaceutical composition according to claim 27, wherein the acetylcholinesterase inhibitor is a dimeric acetylcholinesterase inhibitor.

31. The pharmaceutical composition according to claim 30, wherein the dimeric acetylcholinesterase inhibitor has the structure of formula A-L-B, wherein A and B are acetylcholinesterase inhibitor monomers and may be the same or different, L is an optional linker and, when present, is an alkylene —(CH₂)n—, wherein n is an integer from 1 to 20.

32. The pharmaceutical composition according to claim 31, wherein A and B are independently selected from tacrine and huperzine A.

33. The pharmaceutical composition according to claim 32, wherein the dimeric acetylcholinesterase inhibitor is a tacrine homodimer, i.e., bis(n)-tacrine, as shown below: 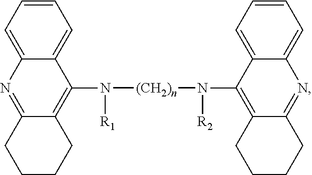
wherein R₁ and R₂ are each independently H or C₄alkyl, and n=2-10.

34. The pharmaceutical composition according to claim 32, wherein the dimeric acetylcholinesterase inhibitor is a huperzine A-tacrine heterodimer, i.e., huperzine A-(n)-tacrine, as shown below: 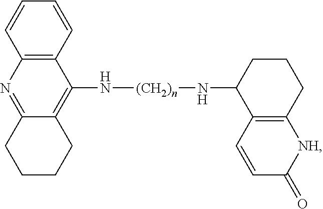
wherein n=4-12.

35. The pharmaceutical composition according to claim 32, wherein the dimeric acetylcholinesterase inhibitor is a huperzine A homodimer as shown below: 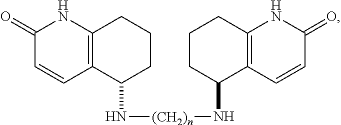
wherein n=10-14.

36. The pharmaceutical composition according to claim 30, wherein the dimeric acetylcholinesterase inhibitor is 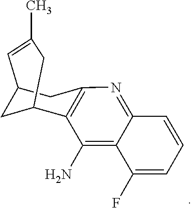 [SMILES: CC1=CC2Cc3nc4cccc(F)c4c(N)c3C(C1)C2]
